(19) United States
(12) Patent Application Publication
(10) Pub. No.: US 2016/0325033 A1
(43) Pub. Date: Nov. 10, 2016

(54) METHOD AND SYSTEM FOR HEART PACING

(71) Applicant: B.G. Negev Technologies & Applications Ltd., Ben-Gurion University, Beer-Sheva (IL)

(72) Inventors: Smadar COHEN, Beer-Sheva (IL); Yoram ETZION, LeHavim (IL); Menahem ROTENBERG, Petach-Tikva (IL)

(21) Appl. No.: 15/109,665

(22) PCT Filed: Jan. 5, 2015
(86) PCT No.: PCT/IL2015/050019

§ 371 Date: Jul. 5, 2016

Related U.S. Application Data
(60) Provisional application No. 61/923,693, filed on Jan. 5, 2014.

(51) Int. Cl. A61M 1/10; A61N 2/02; A61M 1/12

(52) U.S. Cl. CPC A61M 1/1055; A61M 1/1068; A61M 1/122; A61N 2/02

(57) ABSTRACT

A method of pacing a heart of a subject, is disclosed. The heart has a magnetically responsive object therein. The method comprises non-invasively applying to the heart an alternating magnetic field selected to vibrate the object against a wall of the heart such as to effect mechanical stimulation of the heart, thereby pacing the heart.

### RELATED APPLICATION

This application claims the benefit of priority of U.S. Provisional Patent Application No. 61/923,693 filed Jan. 5, 2014, the contents of which are incorporated herein by reference in their entirety.

### FIELD AND BACKGROUND OF THE INVENTION

The present invention, in some embodiments thereof, relates to heart pacing and, more particularly, but not exclusively, to a leadless heart pacing.

Bradycardia is defined as heart rhythm of less than 60 beats per minute at rest. However, symptoms usually manifest only when heart rate goes under 50. Bradycardia may be due to the absence or insufficient electrical impulse from the sinoatrial node (SA) (as in the sick sinus syndrome), or a blockage of the electrical impulse going from the SA to the atrioventricular (AV) node (SA or AV block).

Pacemakers originally were developed for patients with profound bradycardia due to high degree of AV block, who without them usually suffered from cardiac syncope, heart failure and an early demise [Corcoran S J, Davis L M, Cardiac implantable electronic device therapy for bradyarrhythmias and tachyarrhythmias, Heart, lung & circulation. 2012; 21:328-337]. Since that time, device capabilities have evolved to include pacing and defibrillation therapies for tachyarrhythmias and to provide resynchronization therapies for heart failure with the aim of improving quality and, if possible, length of life.

Conventional pacemakers generally includes an electrode connected to a generator that delivers small electrical pulses to the myocardium leading to direct depolarization and action potential generation in some cardiomyocytes. This local excitation is then propagated through gap junctions to adjacent areas and eventually creates excitation of the whole myocardial tissue. Known in the art are pacemakers that have the ability not only to pace the heart but also to sense the necessity for higher rhythm (due to exercise or other stress) and to regulate the electrical pulses accordingly. This feature is achieved by coupling the pacemaker to a sensor that can detect an increase in the body's motion or breathing rate.

Recently, an in man implantation of leadless ultrasound-based cardiac stimulation pacing system has been reported [Auricchio et al., “First-in-man implantation of leadless ultrasound-based cardiac stimulation pacing system: Novel endocardial left ventricular resynchronization therapy in heart failure patients,” Europace: European pacing, arrhythmias, and cardiac electrophysiology: journal of the working groups on cardiac pacing, arrhythmias, and cardiac cellular electrophysiology of the European Society of Cardiology, 2013]. This system includes a piezoelectric chip implanted in the LV so as to harvest ultrasound energy and transduce it into an electric pulse.

Additional background art includes Lee et al., “First human demonstration of cardiac stimulation with transcutaneous ultrasound energy delivery: Implications for wireless pacing with implantable devices,” Journal of the American College of Cardiology, 2007; 50:877-883.

### SUMMARY OF THE INVENTION

According to an aspect of some embodiments of the present invention there is provided a method of pacing a heart of a subject, the heart having a magnetically responsive object therein. The method comprises non-invasively applying to the heart an alternating magnetic field selected to vibrate the object against a wall of the heart such as to effect mechanical stimulation of the heart, thereby pacing the heart.

According to some embodiments of the invention the method further comprising delivering the magnetically responsive object to the heart.

According to some embodiments of the invention the delivering comprises injecting the object to the vasculature of the subject and noninvasively applying an external magnetic field to effect locomotion of the object within the vasculature and into the heart.

According to some embodiments of the invention the delivering comprises invasively implanting the object in the heart.

According to some embodiments of the invention the object is in the cavity of a ventricle of the heart.

According to some embodiments of the invention the object is in an apical portion of the cavity of a ventricle of the heart.

According to some embodiments of the invention the object is in a right ventricle of the heart.

According to some embodiments of the invention the object is in an apical portion of the right ventricle of the heart.

According to some embodiments of the invention the magnetically responsive object comprises at least one of an injectable magnetic nanoparticle, an injectable magnetic microparticle, and a millimeter scale implantable device.

According to some embodiments of the invention the alternating magnetic field is alternating according to a waveform selected from the group consisting of sinusoidal waveform, square wave waveform, triangular waveform, ramp waveform, and any combination thereof.

According to some embodiments of the invention the method further comprising sensing a location of the object in a body of the subject and/or the heart, wherein the application of the alternating magnetic field is responsive to the sensing.

According to some embodiments of the invention the sensing is by a pickup coil operative to generate voltage in response to a change in a magnetic flux.

According to some embodiments of the invention the magnetic object comprises a magnetic material selected from the group consisting of a ferromagnetic material, a ferrimagnetic material and a superparamagnetic material.

According to some embodiments of the invention the magnetic object comprises a superparamagnetic iron oxide.

According to some embodiments of the invention the magnetic object comprises ferromagnetic iron microparticles.

According to some embodiments of the invention the magnetic object comprises a magnetic particle coated by a lipophilic compound.

According to an aspect of some embodiments of the present invention there is provided a system for pacing a heart of a subject, the heart having a magnetically responsive biocompatible object therein. The system comprises: an electromagnet constituted to generate a magnetic field to which the biocompatible object is responsive, and a controller configured to operate the electromagnet to generate an alternating magnetic field selected to vibrate the object once present in the heart against a wall of the heart such as to effect mechanical stimulation of the heart.

According to some embodiments of the invention the controller is configured to operate the electromagnet to generate the alternating magnetic field according to a waveform selected from the group consisting of sinusoidal waveform, square wave waveform, triangular waveform, ramp waveform, and any combination thereof.

According to some embodiments of the invention the system comprises a pickup coil configured for sensing a location of the object in a body of the subject and/or the heart.

According to some embodiments of the invention the system comprises a permanent magnet for applying a static magnetic field in addition to the alternating magnetic field.

According to an aspect of some embodiments of the present invention there is provided a plurality of particles for use in a method for pacing a heart of a subject, the particles being biocompatible, introducible into the heart and responsive to a magnetic field. The method comprises at least some of the operations described herein.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 illustrates a non-invasive pacing technique according to some embodiments of the present invention.

FIG. 2 illustrates an in vitro flow model, according to some embodiments of the present invention. The flow is controlled with a peristaltic pump and circulated in and out the flow chamber. 0.5 ml of different concentrations of magnetic microparticles (C) are injected into a venflon that is connected in the flow line. The flow passes through the flow chamber were magnetic microparticles (MMPs) are subjected to the external magnetic field. The MMPs are indicated by the black blur.

FIG. 3 shows saturation magnetization values of magnetite synthesized in different amounts of ammonium hydroxide or sodium hydroxide, and MMPs containing 48% or 58% magnetite.

FIGS. 4A-B show hysteresis loop of magnetite synthesized in different amounts of ammonium hydroxide or sodium hydroxide (FIG. 4A), and PLA-coated MMPs with different magnetite percentage (FIG. 4B).

FIGS. 5A-B show SEM images of the MMPs (FIG. 5A), and size distribution of the MMP as analysed using SLS (FIG. 5B).

FIG. 6 compares drag force to magnetic force as a function of particle size.

FIGS. 7A-F show absolute magnetic flux density (FIG. 7A), absolute magnetic flux density gradient versus distance from the coil tip (FIG. 7B), and magnetic flux densities (color scale in T) and streamlines for tip diameters of 1, 2, 3 and 4 mm (FIGS. 7C, 7D, 7E and 7F, respectively).

FIG. 8 shows data obtained from the in vitro RV model.

FIGS. 9A-C show cryo-sections of rat hearts were a magnet was positioned on the chest (FIG. 9A), the heart itself (FIG. 9B) and without magnet (FIG. 9C) prior to MMPs injection to the tail vain and 2 minutes later KCL injection to stop the heart. After freezing of the animals with the magnet still on the chest, heart sections were obtained. The dark regions in FIGS. 9A and 9B represents MMPs that were trapped in the RV cavity by the magnetic force. FIG. 9C corresponds to a control experiment in which the magnet was not placed on the chest, so that MMPs did not concentrate in the RV cavity.

FIGS. 10A-B are ECG recordings in rat showing mechanically induced pacing by gently touching the epicardium with a stainless steel tip (FIG. 10A), and by manually attracting with an external magnet, a 1 mm stainless steel bar that is inserted in to the ventricle and is fixed inside the chamber using an attached nylon wire (FIG. 10B). The external magnetic field forces the stainless steel bar on the endicardial surface and induced electrical pacing of the ventricles. N.B.—normal beats. Arrows mark ventricular ectopic beats.

FIG. 11 is a flowchart diagram of a method suitable for pacing a heart of a subject according to various exemplary embodiments of the present invention.

FIG. 12 is a schematic illustration of a system for pacing a heart of a subject, according to some embodiments of the present invention.

FIGS. 13A-D are images of Cryosections of the rat heart after the injection of magnetic microparticles (FIGS. 13A and 13B) and iron microparticles (FIGS. 13C and 13D), with (FIGS. 13A and 13C) and without (FIGS. 13B and 13D) the magnet positioned against the rat chest.

FIG. 14 shows left ventricular pressure in an isolated rat heart (blue) and current as a function of the time in an electromagnet that is directed to the RV (red), as obtained in experiment performed according to some embodiments of the present invention.

FIGS. 15A-D show different types of waveforms used in experiment performed according to some embodiments of the present invention.

FIGS. 16A-H show a current in an electromagnet coil and an arterial pressure in a rat's tail, as obtained in experiment performed according to some embodiments of the present invention.

FIGS. 17A-F show the magnetic flux density intensities (color scale in T) and streamlines for the electromagnet with (FIGS. 17A and 17B) and without (FIGS. 17C and 17D) the metal circuit, and the absolute magnetic flux density (FIG. 17E) and flux density gradient (FIG. 17F) as a function of the distance from the coil tip.

### DESCRIPTION OF SPECIFIC EMBODIMENTS OF THE INVENTION

The present invention, in some embodiments thereof, relates to heart pacing and, more particularly, but not exclusively, to a leadless heart pacing.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

FIG. 11 is a flowchart diagram of a method suitable for pacing a heart of a subject according to various exemplary embodiments of the present invention. The subject is optionally a mammalian subject, preferably, but not necessarily, a human subject. It is to be understood that, unless otherwise defined, the operations described hereinbelow can be executed either contemporaneously or sequentially in many combinations or orders of execution. Specifically, the ordering of the flowchart diagrams is not to be considered as limiting. For example, two or more operations, appearing in the following description or in the flowchart diagrams in a particular order, can be executed in a different order (e.g., a reverse order) or substantially contemporaneously. Additionally, several operations described below are optional and may not be executed.

The method begins at 10 and optionally and preferably continues to 11 at which one or more magnetically responsive objects are delivered to the heart of the subject. The magnetically responsive object can include, but is not limited to, an injectable magnetic particle (e.g., a magnetic nanoparticle, a magnetic microparticle), a millimeter scale implantable device or the like. In some embodiments of the present invention the magnetic object is a magnetic microparticle, and the method delivers a plurality of magnetic microparticles to the heart.

Preferably, the magnetic object is biocompatible and, when a multiplicity of objects (e.g., a multiplicity of particles) is used, they preferably do not aggregate when they are not subjected to a magnetic field. The magnetic objects can comprise any material that is responsive to magnetic field, including, without limitation, ferromagnetic material, ferrimagnetic material and superparamagnetic material.

The magnetic properties of the object originate from the sub-atomic structure of the material. The direction as well as the magnitude of the magnetic force acting on the material when placed in a magnetic field is different for different materials. Whereas the direction of the force depends only on the internal structure of the material, the magnitude depends both on the internal structure as well as on the size (mass) of the material. Ferromagnetic materials have the largest magnetic susceptibility compared to paramagnetic materials.

Superparamagnetic materials consist of individual domains of elements that have ferromagnetic or ferrimagnetic properties in bulk. Their magnetic susceptibility is larger than that of the paramagnetic and similar to their ferromagnetic or ferrimagnetic bulk materials. Ferrimagnetic materials exhibit a magnetic moment that is retained (remanence) after being exposed to an externally applied magnetic field, similar to ferromagnetic substances. Ferrimagnetism is found in ferrites, which are mixed oxides. These materials are crystalline ferric oxide compounds, which resemble ferromagnetic substances in their ability to retain a magnetic field in the absence of an externally applied magnetic field. Although superparamagnetic materials have extremely high magnetic susceptibility as their bulk material, they cannot retain a magnetic moment in the absence of an external magnetic field (no remanence). For this reason, they are preferred materials for biomedical applications.

In some embodiments of the present invention superparamagnetic iron oxide nanoparticles or microparticles (such as, but not limited to, clusters of PLA coated superparamagnetic iron oxide nanoparticles). In some embodiments of the present invention ferromagnetic iron microparticles or nanoparticles are employed.

The particles can optionally and preferably be coated by a lipophilic compound.

As used herein, lipophilic compound, means a compound that has greater solubility in oil than in aqueous medium.

Suitable lipophilic compounds for the present embodiments include, without limitation oleic acid, stearic acid and lipoic acid. For example, the particles can be encapsulated as microparticles with poly-lactic acid coating using double phase emulsion technique.

The size of the particles is preferably selected sufficiently small so as to reduce the risk of embolism and allow easy clearance from the body, and sufficiently large so as to allow them to be captured under the blood flow.

The object can be delivered in more than one way. In some embodiments, the delivery is by an injection. In these embodiments, the object is injected to the vasculature of the subject. An external magnetic field can be applied to effect locomotion of the object within the vasculature and into the heart. In some embodiments, the object is invasively or minimally-invasively implanted in the heart.

The object is preferably delivered to the ventricle of the heart. In various exemplary embodiments of the invention the object is delivered to the apical portion of the ventricle. An advantage of these embodiments is that the geometry of the ventricle allows capturing small objects at its apical portion. In various exemplary embodiments of the invention the object is delivered to the right ventricle (RV), preferably to its apical portion. The advantage of these embodiments is that the RV is the first ventricular chamber receives the object when the object is introduced into the vasculature, so that the likelihood of capturing and maintaining the object in the RV is higher. The RV is also located very close to the chest wall so that external magnetic force can effectively capture the object while it passes in the RV.

The method optionally and preferably continues to 12 at which the location of the object in the body of the subject, or the location of the object within the heart of the subject, is sensed. This can be done, for example, using a pickup coil operative to generate voltage in response to a change in a magnetic flux caused by the object. The voltage generated on a pickup coil correlates with the magnetic moment of the object, the speed of the object and the location of the object relative to the coil. The pickup coil can be placed on or near the skin of the subject, at a position which is in proximity to the heart. The vicinity of the position can be scanned using the pickup coil. The voltage generated in the coil can then be amplified and analyzed so as to remotely sense the location of the object in the body and/or heart.

In various exemplary embodiments of the invention the location is sensed repeatedly so as to monitor the location of the object during pacing. When the object comprises a multiplicity of particles, the monitoring can optionally and preferably include monitoring the number or concentration (e.g., volume concentration) of the objects in the heart. This can readily be achieved by means of the pickup since the voltage generated by the pickup also correlates with the number or concentration of particles.

At 13, an alternating magnetic field is applied, preferably non-invasively, to the heart. The alternating magnetic field is preferably selected to vibrate the object against the wall of the heart. The vibration effects mechanical stimulation of the myocardium and therefore effects pacing by the mechano-electric feedback properties of the myocardium. This provides electrical pacing of the myocardial tissue. The magnetic field can alternate according to any type of time-dependence, including, without limitation, sinusoidal waveform, square waveform, triangular waveform, ramp waveform and any combination of such and/or other waveforms. The alternating magnetic field is preferably selected so as to press the object against the myocardium in a pulsatile manner, thereby generating the mechanical stimulation. When the impact of the mechanical stimulation overcomes a certain threshold electrical pacing evokes and propagates through the entire heart as in the situation of traditional electrical pacing.

The duration and/or spatial dependence of the applied magnetic field is preferably selected so as to prevent at least some of the objects from being carried away from the heart during the pacing. It was found by the present inventors that it is particularly useful to employ a combination of an alternating magnetic field and a static magnetic field, wherein the static magnetic field maintains the object close to or in contact with the myocardium and the alternating magnetic field provides the pacing. In some embodiments, no or reduced static magnetic field is employed. It was found by the present inventors that elimination or reduction of the static magnetic field increases the mechanical stimulation of each magnetic pulse. Thus, these embodiments are useful from the standpoint of mechanical stimulation efficacy.

The magnetic field is typically applied by an electromagnet. An alternating magnetic field can be generated by feeding an AC current through the electromagnet and a static magnetic field can be generated by a permanent magnet and/or by feeding a DC current through the electromagnet. A combination of alternating and static magnetic field can be generated by applying a combination of AC current and DC current. Such a combination can be intermittent or it can be in the form of a complex signal that includes an AC component and a DC component.

In embodiments in which 12 is employed, the application of the alternating magnetic field is preferably turned off during the sensing 12 so as not to generate interferences between the applied magnetic field and the pickup coil. The application of the magnetic field is optionally and preferably responsive to the sensing. For example, the magnetic field can be turned on only upon positive determination that the object is in the heart or in the proper location within the heart (e.g., the RV). Since the object is subjected to the magnetic field, the object acquires magnetization which, together with the mass of the object, provides the object with its magnetic moment. This acquired magnetic moment makes the object detectable by the pick-up coil.

When the method determine that the object is not properly located, the method can loops back to 11 for delivering the object to the proper location.

The method ends at 14.

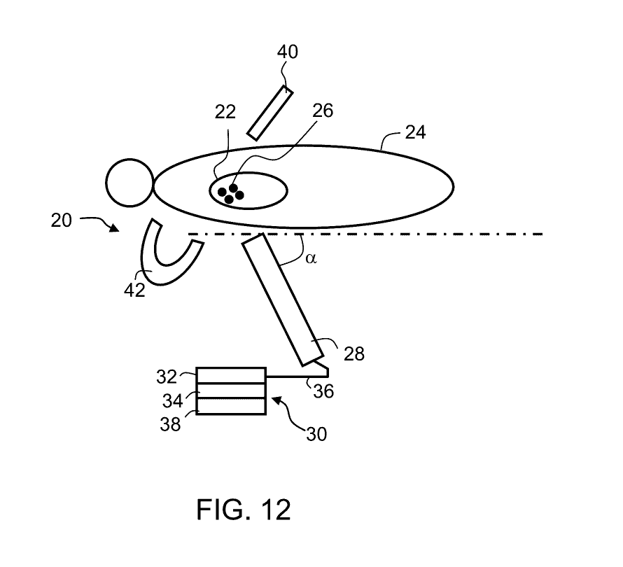

FIG. 12 is a schematic illustration of a system 20 for pacing a heart 22 of a subject 24, according to some embodiments of the present invention. With heart 22, there are one or more magnetically responsive biocompatible objects 26, as further detailed hereinabove. System 20 comprises an electromagnet 28 constituted to generate a magnetic field to which object 26 is responsive, and a controller 30 configured to operate electromagnet 28 to generate an alternating magnetic field selected to vibrate object 26 once present in heart 22 against a wall of the heart such as to effect mechanical stimulation of the heart.

The angle α between electromagnet 28 and the body surface of subject 24 can vary. In some embodiments of the present invention α is approximately 90° but other values for α, e.g., about 30° or about 40° or about 50° or about 60° or about 70° or about 80° are also contemplated.

Controller 30 typically comprises a current generator 32 and a drive circuit 34. Current generator 32 generates an electrical current that is conveyed to electromagnet 28, for example, by an electrical line 36. Drive circuit 34 operates current generator 32 to provide the electrical current according to a predetermined operation protocol. For example, in some embodiments of the present invention drive circuit 34 operates current generator 32 to provide only an alternating current, and in some embodiments of the present invention drive circuit 34 operates current generator 32 to provide a combination of alternating and direct current as further detailed hereinabove. The alternating current can alternate according to any type of time-dependence, including, without limitation, sinusoidal waveform, square waveform, triangular waveform, ramp waveform and any combination of such and/or other waveforms. It is appreciated that the waveform of the current correlates with the waveform of the generated magnetic field, so that drive 34 can select the waveform of the generated magnetic field by selecting the waveform of the current. Drive circuit 34 can comprise, or be connectable to, a CPU 38 configured for operating generator 32 according to the desired pacing of heart 22. CPU 38 can be a dedicated circuitry or part of a general purpose computer.

In some embodiments, system 20 comprises a permanent magnet 42 for applying a static magnetic field to the heart 22. The static magnetic field generated by permanent magnet 42 maintains the object 26 within the heart, as further detailed hereinabove.

In some embodiments of the present invention system 20 comprises a pickup coil 40 configured for sensing the location of object 26 in the body of subject 24. Pickup coil 40 is preferably operative to generate voltage in response to a change in a magnetic flux caused by object 26. Signals from pickup coil 40 can be transmitted to CPU 38 which can analyze the signals so as to remotely sense the location of the object in the heart, as further detailed hereinabove.

The technique of the present embodiments is useful in many applications, particularly, but not exclusively in bradycardia, where intrinsic pacing and/or electrical conduction of the heart is impaired. Conventional emergency techniques which include insertion of a temporary pacing electrode via the vasculature in order to stabilize the patient is usually effective. However, it was realized by the present inventors that this procedure may take time and in many cases can be done only under fluoroscopy, which may not be available acutely.

As a bridging alternative, the clinician can use transcutaneous pacing (external pacing) to deliver pulses of electric currents through the patient's chest that would provoke heart pacing until intravenous electrode insertion can be applied. Although external pacing is a life saving procedure and is the current standard of care in such situations, it was realized by the present inventors that this technique is not without certain operative limitations that would best be avoided. For example, this technique is painful and in most cases necessitates the use of sedative or anesthetic agents, which can further impair the critical hemodynamic condition of the patient. In addition, external pacing is applicable only for short periods of time (e.g., several hours), since it can cause substantial damage to surrounding cells and tissues.

The technique of the present embodiments allows for effective, painless non-invasive pacing, and can therefore serve as a bridging treatment, from the time of admission to the emergency room until a definitive procedure is effectively performed.

Another situation where the technique of the present embodiments is advantageous is in patient suffering from tachycarrhythmia. In such conditions, pacing can be used to acutely stop the arrhythmia. For example, in modern implantable defibrillator (ICD) the device can be programmed try a protocol of anti-tachycardia pacing (ATP) for several seconds, before it actually gives an electric shock to induce cardioversion/defibrillation.

Another application of the technique of the present embodiments is for diagnostic pacing in the electrophysiological laboratory. In the electrophysiological laboratory, clinicians introduce electrodes and pace the heart in various locations in a way that can to provoke arrhythmias. Such procedure is termed “programmed electrical simulation” (PES) and is practiced for the purpose of determining the tendency for arrhythmia induction in the patient's heart. Since there are specific locations that are hard to access with electrodes (such as the left side of the heart), the technique of the present embodiments may be utilized to pace these locations non-invasively.

Another application of the technique of the present embodiments is for permanent leadless pacing, where traditional lead pacing suffers from limitation associated with the leads such as: lead failure, the necessity of lead replacement, children related complication (due to the risk of venous thrombosis and to the expected growth), anatomical positioning limitations etc.

The physiological mechanism that is exploited by the method and system of the present embodiments will now be explained in detail.

Mechanical effects on heart rhythm are known, and documented cases include both arrhythmogenic and pro-rhythmic consequences of mechanical stimulation. The intracardiac pathway that leads from changes in the cardiac mechanical environment to altered electrical activity is referred to as mechano-electric feedback (MEF). The occurrence of ectopic beats during cardiac catheterization is understood to be a reliable indicator of the approach of the catheter-tip to the endocardium, which is a non-expected and useful utility of MEF. Cardiac cells respond to mechanical stimuli almost as readily as they do to electrical stimuli.

Some embodiments of the present embodiments are based on the observation that ventricular excitation may be evoked by precordial thumps. The molecular mechanism linking mechanical and electrical activity correlates with the activity of stretch activated ion channels, whose pharmacological block was found to be sufficient to terminate mechanically promoted atrial fibrillation as well as ventricular rhythm disturbance. In addition, there are many coupling mechanisms and modulators, at microscopic and macroscopic levels. These involve changes in calcium ion handling, auto and paracrine messenger cascades, homo- and heterotypic cell interactions, as well as spatial and temporal variations in wall stress.

Mechano-sensitive channels (MSC) or stretch-activated channels (SAC) have gating rates sensitive to mechanical stress in their environment. The mechanical sensitivity can be in addition to more traditional stimuli. That environment may include the lipid bilayer, the cytoskeleton and the extracellular matrix (ECM). Mammalian SACs occur in two classes: those associated with specialized receptors, such as the cochlea, muscle spindle and pacinian corpuscles, and those in all other cells notably the heart.

No generic molecular structure associated with SACs is known. Still, they share a property of a significant change in channel dimensions between the close and open states; open SACs are larger in size (in the plane of the membrane) than closed SACs. This size does not refer to the diameter of the pore but to the outer physical dimensions where the proteins meet the lipids that bear the tension. The free energy difference AG between the open and closed states is proportional to the tension T and to the difference in planner area AA in both states according to EQ. 1:

ΔG=TΔA (EQ. 1)

This relation is the main cause of stretch induced SAC opening, although some local effects of curvature may also contribute to this character. The most extensive study on SAC kinetics was done with TREK-1, a K₂ₚ (potassium two pore channel) that is expressed in fetal neurons and in cardiomyocytes. The K₂ₚ is a diverse family which its membrane topology is unique compared to other known potassium channels and is characterized by four trans-membrane domains (DM1-DM4) and two pore forming domains (P1 and P2). Membrane stretch reversibly induce TREK-1 channel opening in both cell attached and excised inside-out configurations.

Several different types of mammalian mechano-gated ion channels and receptors, however, have been identified and cloned. In addition to the K₂ₚ family, a number of voltage or ligand-activated cardiac ion channels have been found to be modulated by the mechanical environment, including the ATP dependant potassium channel, K⁺_{ATP}. Another group of SACs are ion channels activated by change in cell volume (volume activated channels, VAC). VACs are not directly activated by changes in cell length or by local membrane deformation, but require an increase in cell volume.

The epithelial sodium channel (ENaC) belongs to a family of eukaryotic ion channels which share a motif of two transmembrane domains connected to an extracellular loop and cytosolic C and N-termini. The ENaC is expressed on the apical surface of a variety of epithelial cells including the kidney, lung, colon and heart, where it is involved in maintenance of body salt and water homeostasis by absorption of sodium ions.

Given the wide variety of SACs, no universal blocker was found to date. However, general pharmacologic effectors include non-specific lanthanides, such as La³⁺ and Gd³⁺. Gd³⁺ sensitivity, while often used as a signature of SAC channels, is less reliable since it has significant reactivity with other channels. In addition, Gd³⁺ rapidly precipitates many physiological anions including PO⁴3-, HCO³—, and proteins, so it cannot be used in physiological conditions. Amiloride and cationic antibiotics such as streptomycin have been used to block SAC, but these drugs are non-specific. SAC may also be sensitive to specific ion channels reagents such as tetrodotoxin and diltiazem.

The discovery of GsMTx-4, a specific inhibitory peptide, isolated from tarantula venom and now commercially available, has provided an important novel tool to study SAC effect in cellular and multi-cellular settings. This peptide blocks SAC in variety of cell types, including chick heart, rat heart and astrocytes and rabbit, dog and sheep ventricular cells.

Some embodiments of the present invention relates to pacing using magnetic particles. The versatile intrinsic properties of magnetic particles allows their use in numerous medical applications such as: magnetic fluid hyperthermia, where selective thermal ablation of tumors is achieved through heating of tumor-localized magnetic particles exposed to high frequency magnetic field; tissue engineering, where particles can be used in remote actuation for control of cellular behavior enabling development of functional tissue or to provide means for a patterned cell assembly and facilitated seeding of tissue engineered scaffold with functional cells; MRI, where magnetic particles are used as contrast agents; and probably the most investigated feature-localization of therapy, where magnetic carriers associated with drugs, nucleic acid or loaded within cells or liposomes can be directed or guided by means of a magnetic field gradient towards certain biological targets.

In various exemplary embodiments of the invention the particles are made of a superparamagnetic material. These particle are ferromagnetic material particles that are sufficiently small (two to several tens of nm, depending on the material) so they are consisted of a single domain of magnetic moment. This domain is free to randomly flip direction, thus the magnetization of the particles of the present embodiments is preferably zero in average, although they can carry a high magnetic moment as the bulk material. Representative example of a magnetic material suitable for the present embodiments include, without limitation, magnetite (Fe₃O₄), which is a ferrimagnetic material that may be easily syntheses to crystals with the size of 8-12 nm.

To capture a magnetic particle in a specific site, a magnetic attraction force that is larger than the drag force applied on the particle by the blood flow is applied. The force applied by a magnetic field on a magnetic particle is described by EQ. 2.

F=(m·∇V)B (EQ. 2)

where F is the force, m is the magnetization, B is the magnetic field, and V is the vector differential operator. Throughout this specification, vector quantities are denoted by underlined symbols. The force as defined by EQ. 2 above increases with the magnetization m and the gradient of the magnetic field B. For given magnetic field B the magnetization m depends on the mass of the particle, so that the magnetic force is proportional to the third power of the particle's radius.

The drag force applied by the blood flow over a spherical object can be approximated by Stokes' law as described in EQ. 3.

F_{d}=6πμRv (EQ. 3)

where F_{d} is the drag force, μ is the viscosity of the blood, R is the radius of the particle and v is the velocity of the flow. From EQ. 3 one sees that the drag force is proportional to the radius, so that as the particle size increases the magnetic force applied on it becomes more dominant over the drag force applied by the blood flow.

The present embodiments localize a sufficient amount of magnetic particles in the heart, and generate a pulsed attraction of these particles by an alternating magnetic force, so as to apply mechanical stimuli for heart pacing.

The pacing of the present embodiments can be used in cases of heart block leading to symptomatic bradycardia, and can replace the need for acute external pacing followed by transvenous insertion of a temporary pacing electrode until a permanent heart pacer is finally transplanted.

The particles employed by the present embodiments are optionally and preferably magnetic microparticles (MMPs). In various exemplary embodiments of the invention the particles comprise a magnetic material, preferably but not necessarily superparamagnetic material, e.g., magnetite. The particles are optionally and preferably coated with the biocompatible material, such as, but not limited to, poly lactic acid (PLA), which is an FDA approved biocompatible material. The particle of the present embodiments can be synthesized in a size range that allows their flow in circulation, preferably without causing embolism. The size of the particles can be selected to allow their localization to the RV upon applying an external magnetic field.

The magnetic field can be generated by an external magnet, that can be a permanent magnet or, more preferably, an electromagnet that typically includes a coil and a core. The magnet can be designed using a computerized model as known in the art. The particles can be guided into the RV by means of a magnetic force applied by a magnetic field generated by the same magnet that induces the pacing or by a different magnet. The mechanical stimulation of the heart can be effected by an alternating magnetic field in order to provoke MEF-induced pacing.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### Examples

Reference is now made to the following examples, which together with the above descriptions illustrate some embodiments of the invention in a non limiting fashion.

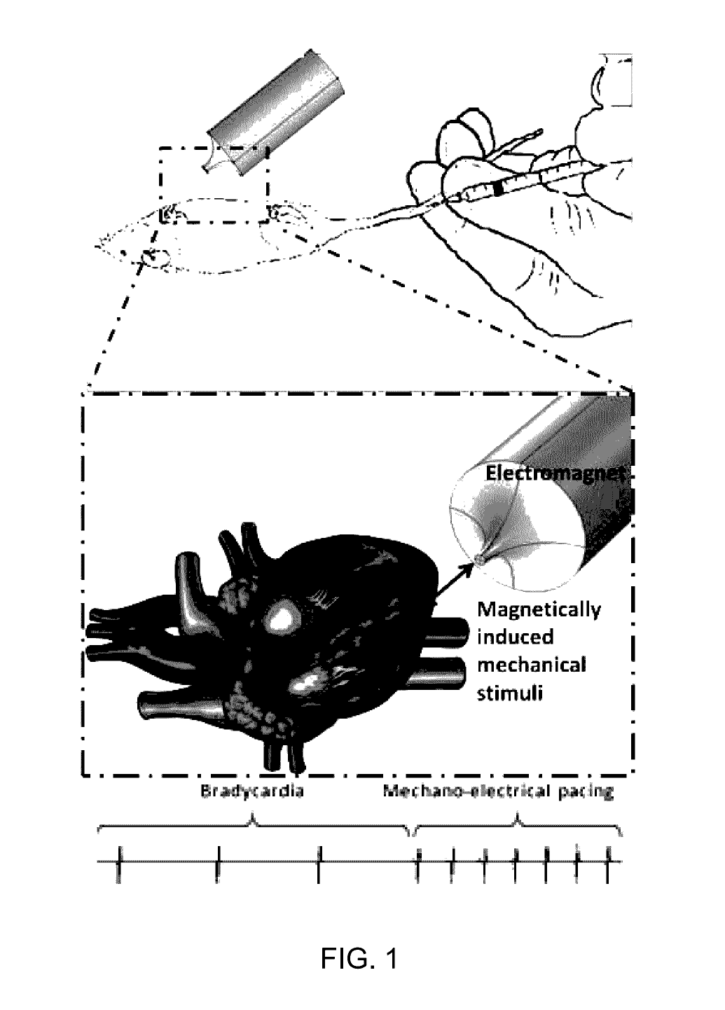

FIG. 1 illustrates a non-invasive pacing technique according to some embodiments of the present invention.

### Synthesis and Characterization of Magnetic Microparticles (MMPs)

The process consisted of three stages: preparation of the magnetite nano particles, their modification with oleic acid and finally their encapsulation in PLA microparticles.

### Preparation of the Magnetites:

Briefly, ferric chloride hexahydrate and ferrous chloride tetrahydrate (455 mg and 168 mg, respectively) were dissolved in 5 ml degassed (by argon bubbling) double-distilled water (DDW) and magnetites were obtained by alkaline precipitation with different amounts of 2 M sodium hydroxide (2.5, 3.36 and 5 ml) or concentrated (28-30 wt %) ammonium hydroxide solution (0.66, 0.71, 0.76, 0.81, 0.86 and 0.91 ml). The magnetites were magnetically separated, rinsed twice in DDW and dried under vacuum. To determine the best precipitation process, the magnetic properties of the magnetites were analyzed using a vibrating sample magnetometer (VSM, MicroSense, LLC, USA). The value of their magnetization saturation at 10 kOe was determined and the superparamagnetic property was verified according to its hysteresis loop.

The magnetites were then prepared by precipitating a solution of ferric chloride hexahydrate and ferrous chloride tetrahydrate (11.6 g and 4.3 g, respectively) in 350 ml degassed DDW by 20 ml of ammonium hydroxide. This suspension was heated to 80° C. under argon bubbling for 5 min.

Modification with Oleic Acid

1 ml of Oleic acid (OA) (Sigma, USA) was added and the suspension was stirred vigorously while kept on 80° C. for 25 min. When the stirring was stopped, a black tar of lipophilic magnetites was spontaneously separated and settled down on the bottom of the flask. The supernatant aqueous phase was aspirated and the pelleted magnetites were rinsed twice by DDW to remove salts and ammonium hydroxide residues, and twice by ethanol to remove excess non-reacted OA.

### Encapsulation in PLA Microparticles

Two formulations of MMPs were synthesized, consisting of 58 or 48 wt % of magnetite (58% MMPs or 48% MMPs, respectively). The lipophilic magnetites (560 mg) were dispersed in a 3 ml dichloromethane (DCM) with dissolved PLA (400 mg for 58% MMPs, or 600 mg for 48% MMPs). The organic phase was emulsified in 8 ml of 1.5% (w/v) polyvinyl alcohol (PVA, 27 kDa, 98% Hydrolyzed) in DDW saturated with DCM by vortexing for 1 min under maximum power, and poured into 200 ml of 0.2% (w/v) PVA and stirred for 5 min. Then 12 ml of isopropyl alcohol was added and the organic solvent was evaporated by stirring in a chemical hood for 30 min. The obtained MMPs were passed through a 106 μm filter to remove large aggregates and lyophilized. Lyophilized MMPs were kept at room temperature in a desiccator and re-suspended in Phosphate buffered saline (PBS) before use. In addition to MMPs I synthesized PLA encapsulated magnetite nano particles by using 2 min of max power of probe sonication as the emulsion technique. Their size range was determined by visualizing them using a transmitting electron microscopy (TEM).

MMP size measurements were performed using static light scattering (SLS, FRITSCH ANALYSETTE 22 MicroTec plus, Idar-Oberstein, Germany) and verified by Olympus light microscope (BX61, Motorized System Microscope). The MMP morphology was further investigated by using scanning electron microscopy (SEM); MMP suspension in DDW was deposited on glass slide and then vacuum dried, coated by 5 nm gold layer for contrast and visualized using SEM. The magnetic properties of MMPs were obtained from the hysteresis curves obtained by vibrating sample magnetometer (VSM, MicroSense, LLC, USA).

In addition to MMPs, PLA encapsulated magnetite nano particles were synthesized by using 2 min of max power of probe sonication as the emulsion technique. Their size range was determined by visualizing them using a transmitting electron microscopy (TEM).

### Theoretical Calculation of Particle Size

To assess the size range of the MMPs to be synthesized the magnetic and drag force applied on MMP under a certain fluid flow was calculated by EQs. 2 and 3, respectively. The assumptions taken under consideration were MMP of spherical morphology, particle saturation magnetization is 40 emu/g and that magnetic field is large enough to induce this magnetization (B>0.5 T), magnetic field gradient of 20 T/m, laminar flow (Re<<10⁵), velocity is 8 cm/s and viscosity is 3.5·10⁻³ Pa·S. The forces were calculated for different MMP diameter ranging from 1-100 μm using Excel® software and compared.

### Electromagnet Design

### First Design

The electromagnet was made out of a coil were a steel bar was inserted into its core. The coil was an air core solenoid (PASCO scientific, Roseville, Ca) 5.5 cm diameter, 14.5 cm long and consisted out of 560 turns. The core was fabricated out of 1040 steel in the university workshop. The core body was fitted to the coil and the edge was narrowed to a 4 mm tip in order to concentrate the magnetic field stream lines. The magnetic field generated by the electromagnet was simulated using COMSOL Multiphysics software (COMSOL Inc., Burlington, Mass.). The simulation was performed by solving equations 4 and 5:

∇×H−σv×B=Jₑ (EQ. 4)

B=∇×A (EQ. 5)

where H is the magnetic field (A/m), B is the magnetic flux density (T), Jₑ is the current density (A/m²), σ is the conductivity, v is the velocity and A is the magnetic vector potential. The properties of the steel, air, and copper wire were obtained from the software material library while the relative permeability and permittivity of the steel was 100 and 1, respectably.

### Second Design

The electromagnet was made out of a coil were a 13 cm permendur (Goodfellow, UK) bar was inserted into its core. The coil was comprised of 900 turns around the bottom 10 cm, and the 3 cm out of the coil were narrowed to a 4 mm tip in order to concentrate the magnetic field stream lines. A metal circuit was added to further induce the stream line concentration.

The magnetic field generated by the electromagnet was simulated in the same way, with changing the permendur permeability and permittivity to 1000 and 100, respectably.

The general concept of the designed electromagnet is that by inserting the permendur core that narrows at its tip into the coil the magnetic field stream lines are concentrated and the magnetic flux density dramatically increases.

### In Vitro RV Model

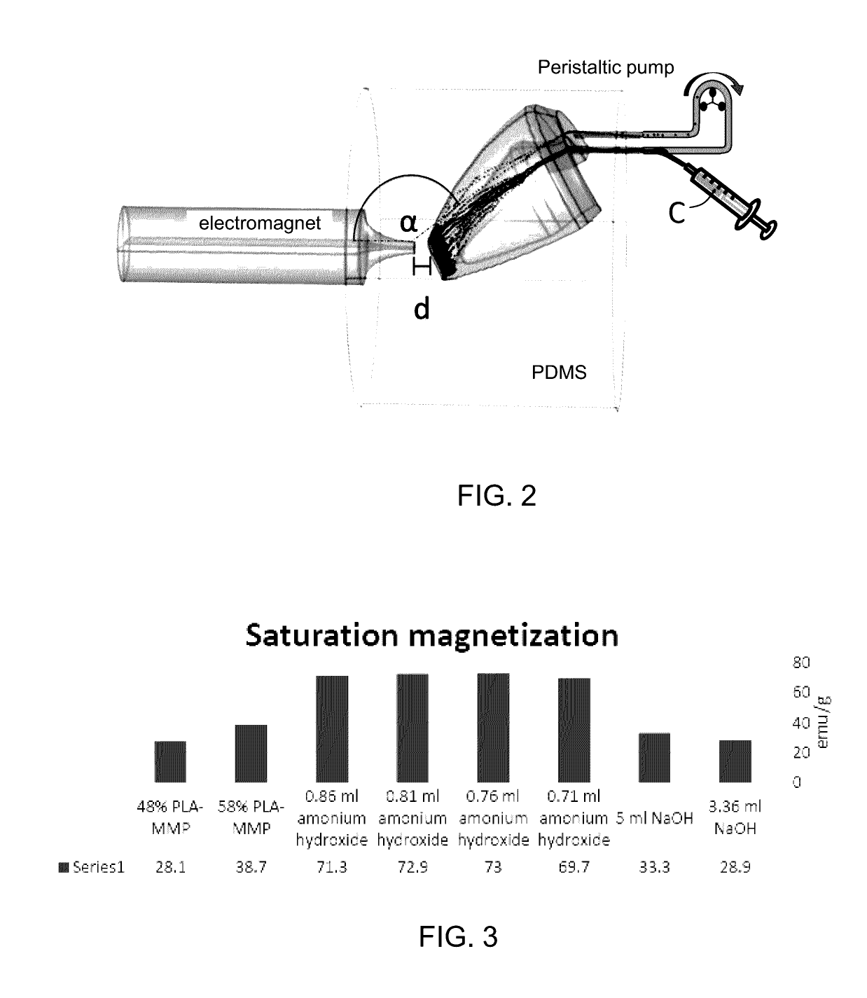

A capture study has been performed in an in vitro fluid flow system model of the RV cavity. The model system was consisted of a flow chamber fabricated from a polydimethylsiloxane (PDMS) template that was cured around a Teflon piece fabricated in the shape of a rat RV. The morphology had the cross-section of a crescent, 2 mm wide, 7 mm long at the top (were the valves are) that narrows down to a round edge (the apical portion) while the ventricle wall is 12 mm. After the template was cured two silicon tubes (inlet and outlet) were inserted in a location that mimicked the location of the valves in animal heart. Then the template was pressed against the electromagnet tip as illustrated in FIG. 2.

In order to mimic blood viscosity, 40% glycerin in PBS was perfused through the flow chamber at 45 ml/min. Different amounts of MMPs were injected while the electromagnet was set on. After 1 min of perfusion the pump was turned off, then the reservoir was switched to a pre-weighted clean one with fresh 40% Glycerin in PBS, the electromagnet was switched off and the pump turned back on to wash out the MMPs to the clean reservoir sitting on a magnet. After 1 min, the reservoir was disconnected, the MMP were washed with DDW and then dried by vacuum. After removing the water the reservoir was weighted again and the weight of the captured MMP pellet was subtracted.

### MMPs Localization in a Rat RV

To verify the feasibility of capturing MMPs in the RV using the electromagnet several experiments are performed, as follows.

### Ultrasound Live Imaging of MMP Capture in the Heart

Rat is anesthetized and a neodymium n52 magnet (cylinder, axially magnetized, 13 mm diameter and 13 mm long) is gently pressed against the chest. A commercially available ultrasound system (vivid-7, GE-Vingmed, Milwaukee, Wis.) is used to visualize the RV using a 11 MHz transducer. 50 mg of 58% MMPs suspended in 0.5 ml PBS is injected through the tail vein and the particle movement in the RV is recorded. PLA encapsulated magnetite nano particles (200-300 nm) are also injected and recorded by ultrasound for comparison.

### Open Chest Model

A rat is anesthetized and its chest is opened. A neodymium n52 magnet (3.2 mm diameter and 6.35 mm long) is sutured against the apical portion using 6.0 nylon suture. Then MMPs at an amount determined by the in vitro flow model are injected into the rat tail vain and captured in the RV by the neodymium magnet. Thereafter, the rat is sacrificed by injection of KCl solution while the magnet is still in place and the chest is immediately frozen by dry ice followed by extraction of the magnet and further freezing of the whole body of the rat in a −70° C. freezer. The frozen body is thawed and the heart is fixed in a OCT block and is cryo-sectioned to visualize the MMPs in the ventricles.

### Close Chest Model

A rat is anesthetized and the electromagnet (set on DC in order to generate permanent magnetic field) is placed or a neodymium n52 magnet against the rat's chest. Then, MMPs at an amount determined by the in vitro flow model are injected into the rat tail vein and captured in the RV by the external magnet. Then the rat is be sacrificed by injection of 0.5 ml KCl solution, and the body is frozen. The frozen heart is thawed and fixed in a OCT block and the heart is cryo-sectioned to visualize the MMPs in the ventricles.

In another experiment, MMPs or iron microparticles (Sigma Aldrich) were injected into the rat tail vein. The microparticles were injected while an N42 neodymium magnet was positioned against the rat heart. After the particles were captured in the RV (about 1 min) the rat heart was stopped by KCL injection and the rat was frozen while the magnet is still positioned against the heart. FIGS. 9A-C show cryo-sections of rat hearts were the magnet was positioned on the chest (FIG. 9A), the heart itself (FIG. 9B) or without magnet (FIG. 9C) prior to MMPs injection to the tail vain and 2 minutes later KCL injection to stop the heart. After freezing of the animals with the magnet still on the chest, heart sections were obtained. The dark regions in FIGS. 9A and 9B represents MMPs that were trapped in the RV cavity by the magnetic force. FIG. 9C corresponds to an experiment in which magnet was not placed on the chest MMPs did not concentrate in the RV cavity.

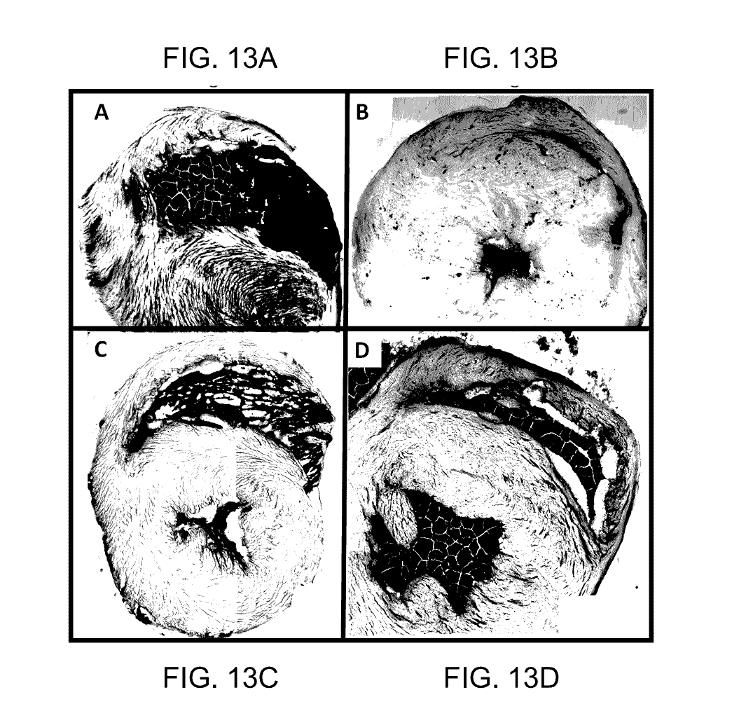

In FIGS. 13A-C cryosections of the heart were performed to visualize the particles located in the RV in a close chest mode only. FIGS. 13A-C are images of Cryosections of the rat heart after the injection of magnetic microparticles (FIGS. 13A and 13B) and iron microparticles (FIGS. 13C and 13D), with (FIGS. 13A and 13C) and without (FIGS. 13B and 13D) the magnet positioned against the rat chest. As shown, when the magnet was positioned against the heart, the particles were localized in the RV (FIGS. 13A and 13C). Without the magnet positioned against the heart the RV was vacant of particles (FIGS. 13B and 13D).

### Non-Invasive Temporary Mechanical Pacing

The ability to induce heart pacing using magnetically induced mechanical stress was verified in an experiment.

### Permanent Magnet Induced Pacing

A rat was anesthetized and peripheral Electrocardiography (ECG) was continuously recorded. Following left lateral thoracotomy, a stainless steel bar (1 mm in length) attached to a 6.0 nylon wire was inserted into the RV through the apical portion. The wire was used to keep the metal bar from flowing away along with the blood flow. After we verified that the myocardium sealed over the wire and no bleeding was noted we used a neodymium n52 magnet (cylinder, axially magnetized, 13 mm diameter and 13 mm long) that was manually positioned against the chest rapidly in order to generate a pulsatile magnetic attraction on the needle and to induce premature ventricular contractions (PVCs). The magnet was removed and re-positioned rapidly several times. The pacing, triggered PVCs were recorded using an ECG amplifier (Nihon Kodhen, RMC1100) and stored on a PC using Labview designed software and an A/D converter (PCI-6024E, National Instruments, Austin, Tex., USA).

### Electromagnet Induced Pacing

Open Chest

An electromagnet is placed against the open chest and a sinusoidal wave of magnetic field, ranging from 0 to 1 T (at the end of the electromagnet tip), is generated. The magnetic force applied on the needle generates pulsatile mechanical stress on the endocardium. The pacing, triggered by the metal bar, is recorded using in the peripheral ECG as described above.

Langendorff Perfused Heart

In an experiment performed by the present inventors, a Langendorff perfused heart model was used. In this model, the rat heart is perfused with oxygenated buffer so its contractility remains outside the body. IMPs were introduced using a syringe directly into the RV. Pacing was then evoked by mechanical stimulation of the heart. The mechanical stimulation was generated by applying pulses of magnetic fields; the magnetic field caused the magnetic particles to be pushed towards the RV wall. In order to see the effect of the mechanical stimulation on the heart rhythm, the pressure in the left ventricle (LVP) was measured using a latex balloon positioned in the left ventricle and connected to a pressure transducer, while applying the magnetic pulses.

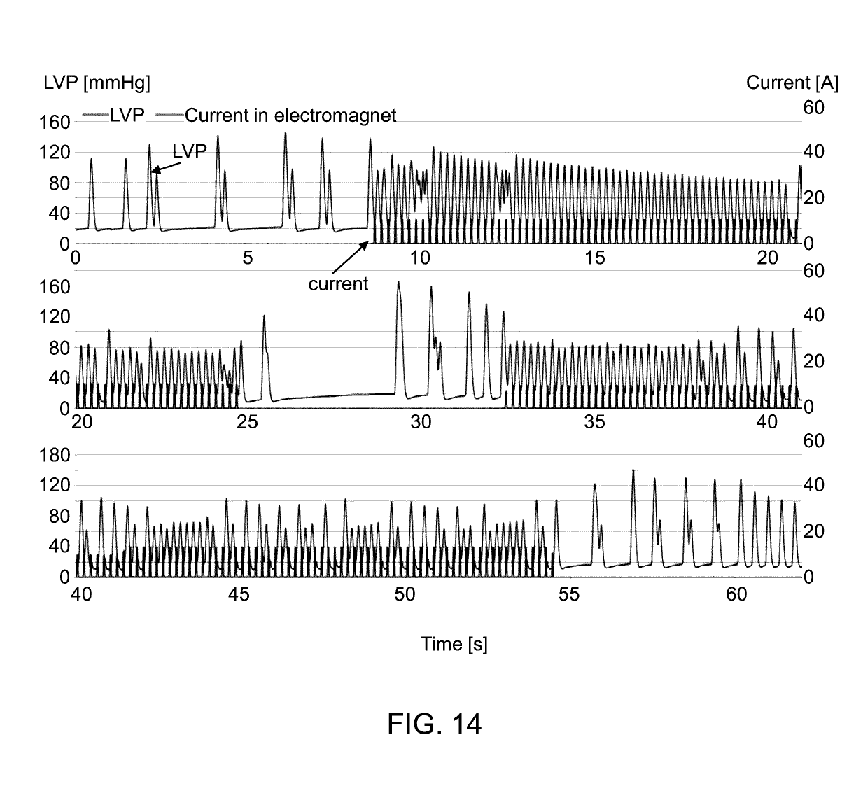

FIG. 14 shows the LVP and the current in the electromagnet as a function of the time. Each pulse in the LVP represents a heart contraction, and each pulse in the current line represents a magnetic pulse. As shown, when no magnetic pulses were applied, the heart rhythm was extremely slow due to the removal of the right atrium (RA) and mechanical induction of atrio-ventricular block. When mechanical stimulation was applied according to the present embodiments, the heart rhythm immediately synchronized with magnetic pulses. In addition, when the mechanical stimulation was terminated, the heart rhythm immediately returned to its slow rhythm. Since there is no blood flow in the Langerdorff perfused heart model, the particles remained in the RV.

Rat Heart

A rat is anesthetized and connected to ECG electrodes. The electromagnet is placed against the chest. The function generator connected to the coil is set to operate in a direct current (DC) manner to give an electromagnet output of permanent magnetic attraction. Then, MMPs at an amount determined by the in vitro flow model are injected into the rat tail vain and captured in the RV by the electromagnet. After a pellet of MMP is captured in the RV the function generator is operated to generate alternating current (AC) of a sinusoidal wave in order to give an electromagnet output of sinusoidal magnetic attraction. This attraction generates the mechanical stress that is transduced into heart pacing.

In experiments performed by the present inventors, the tail artery was cannulated for arterial pressure (AP) measurement. Magnetic particles were injected into the tail vain, while the electromagnet was positioned against the heart. After the magnetic particles were localized in the RV, magnetic pulses were applied using the electromagnet.

Following is a description of the various configurations and settings that were employed in the experiments.

In some experiments, the electromagnet included a metal loop forming a magnetic circuit for amplifying the magnetic induction, and the magnetic induction gradient. Experiments performed with and without the metal loop.

The angle α between the electromagnet and the rat (see FIG. 12) was varied among different experiments. In the present example, the values α=45° and α=90° were used.

Two different locations of the electromagnet relative to the rat's chest were tested. In some experiments it was located at the left side of the chest, and in some experiments it was located at the right side of the chest. In all experiments the electromagnet was pointed to the apical portion of the rat's heart.

In some experiment a permanent magnet (see 42 in FIG. 12) was used to apply a static magnetic field on the particles so as to retain them in the in the RV. The angle between the magnetic field generated by the permanent magnet and the magnetic field generated by the electromagnet was approximately 90°.

Two different types of particles were used. Magnetite microparticles coated with PLA (MMP) and iron microparticles (IMP).

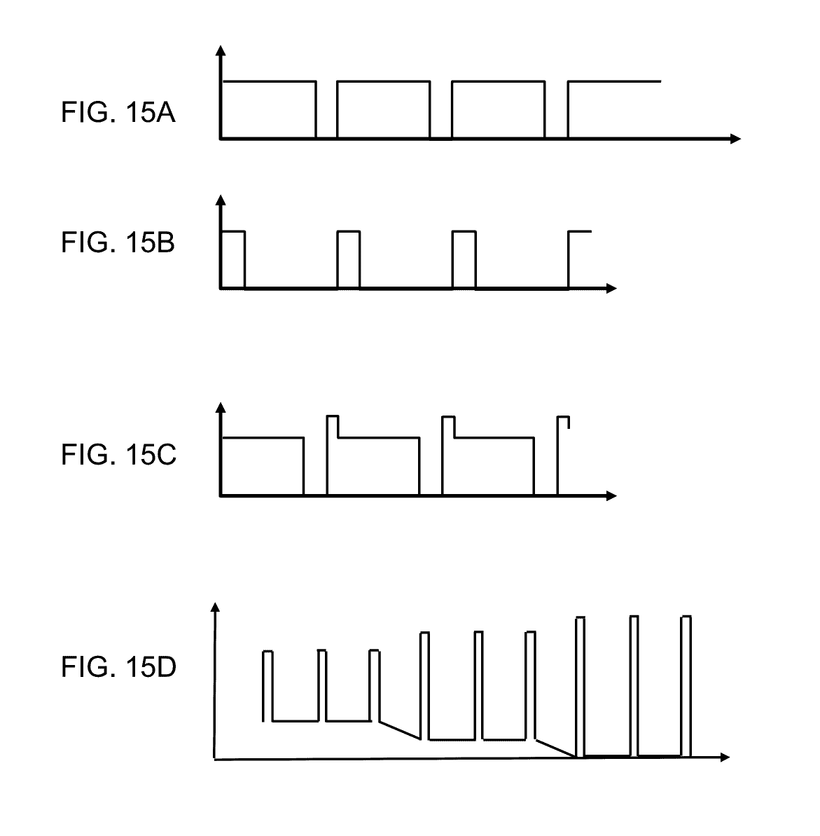

Four different types of waveforms-were employed. These are illustrated in FIGS. 15A-D. A first type, referred to below as a “high duty waveform” was a square wave in which the magnetic field was toggled between a constant value and a zero value at a duty cycle of 80-90% for the constant value and 10-20% for the zero value (FIG. 15A). A second type, referred to below as a “low duty waveform” was a square wave in which the magnetic field was toggled between a constant value and a zero value at a duty cycle of 10-20% for the constant value and 80-90% for the zero value (FIG. 15B). In a third type, referred to below as “DC+AC”, a constant magnetic field followed by a zero magnetic field which followed by a short pulse of magnetic field, at a duty cycle of 70%:20%:10% respectively (FIG. 15C). In a fourth type, referred to below as “Added AC” an alternating current is added to a direct current, resulting in magnetic pulses with elevating levels field over 10-20% of the time, and low magnetic field over 80-90% of the time.

This Example describes eight experiments. The configuration and settings for each experiment are summarized in Table 1.

TABLE

| | | | Electro- | | | |
|---|---|---|---|---|---|---|
| Experiment | Metal | | magnet | Permanent | | |
| No. | Loop | α | Location | magnet | particles | Waveform |
| 1 | YES | 90 | Left | NO | IMP | Low duty |
| 2 | YES | 90 | Left | NO | IMP | DC + AC |
| 3 | NO | 45 | Left | NO | IMP | Low duty |
| 4 | NO | 45 | Left | NO | IMP | High duty |
| 5 | NO | 45 | Left | NO | MNP | Low duty |
| 6 | NO | 45 | Right | NO | IMP | Low duty |
| 7 | NO | 45 | Left | NO | IMP | Added AC |
| 8 | NO | 45 | Left | YES | IMP | Low duty |

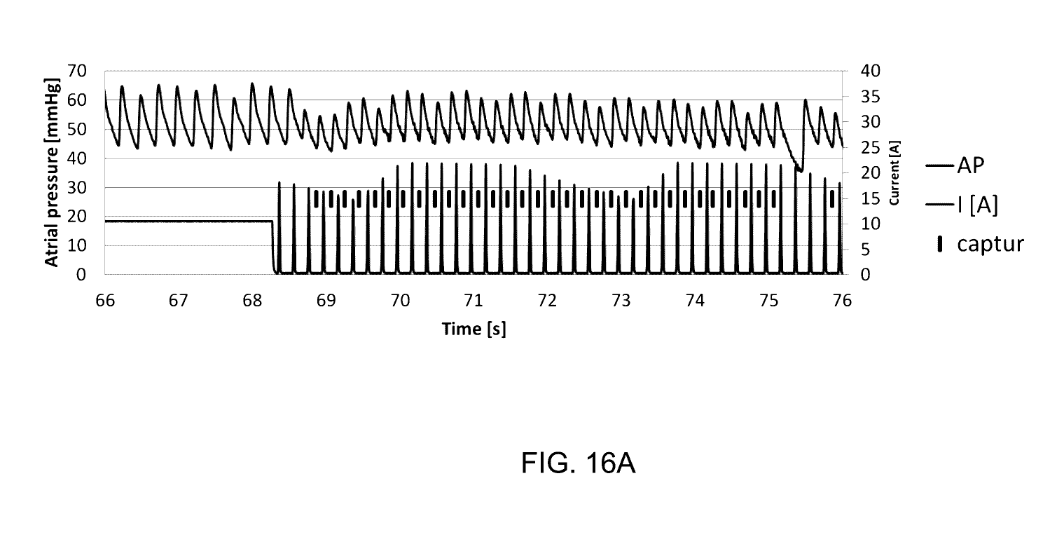

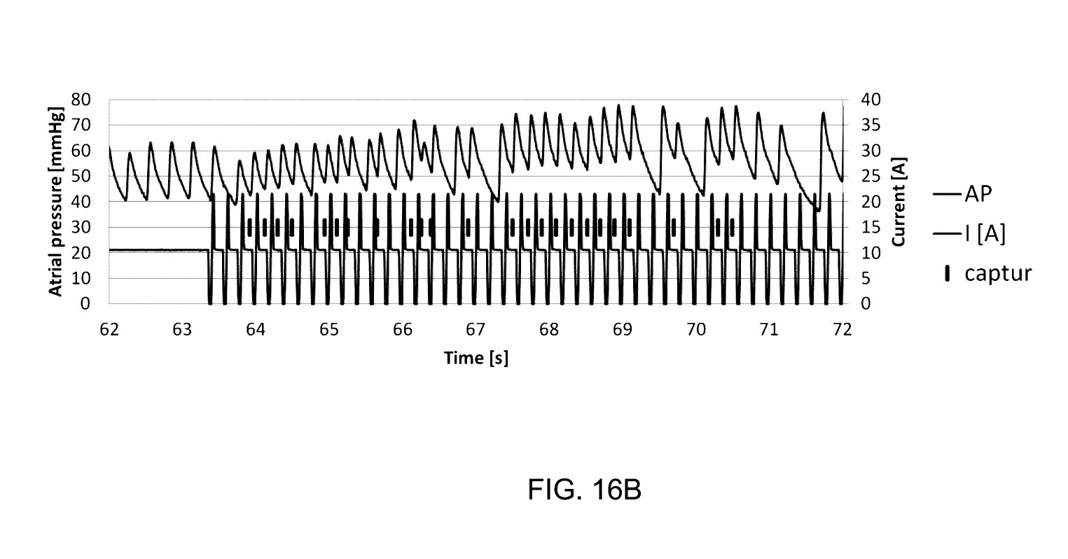

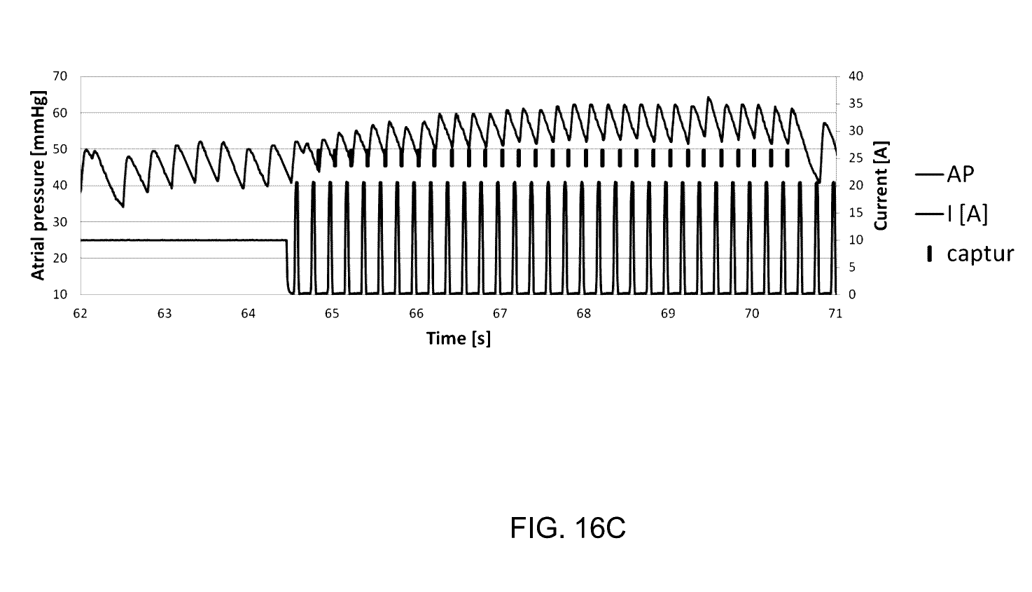

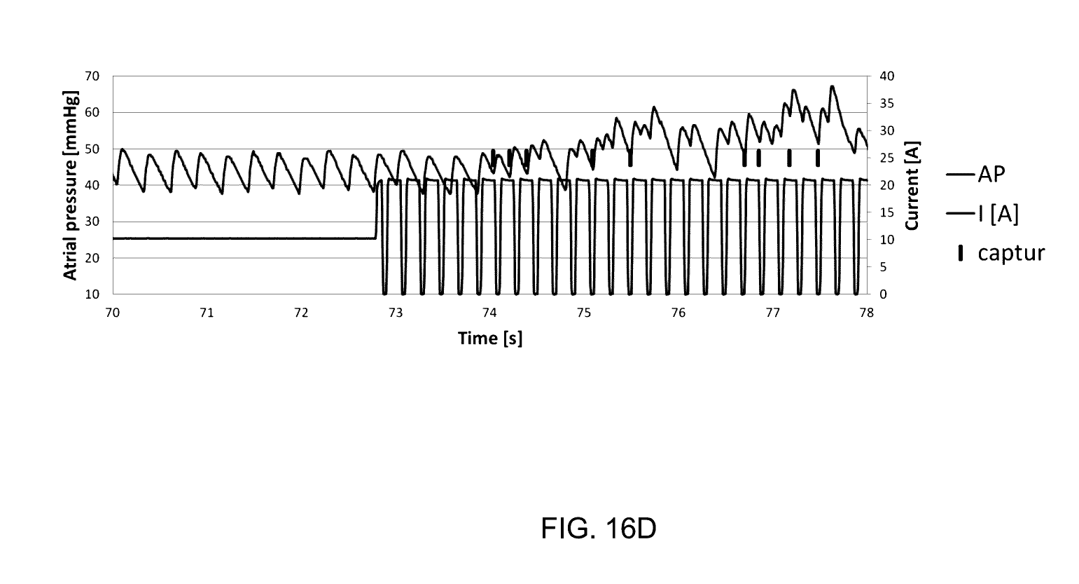

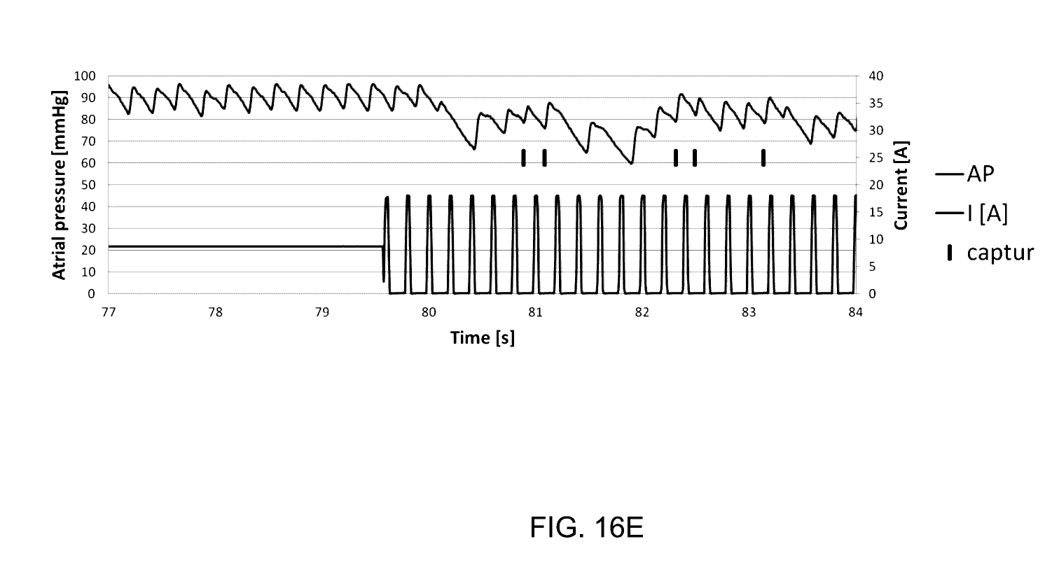

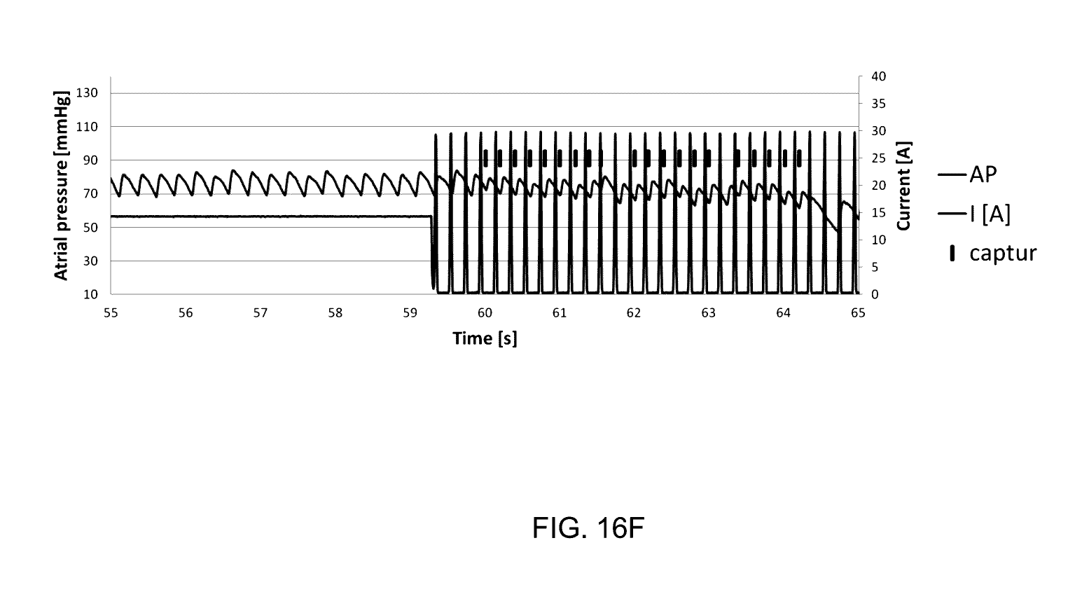

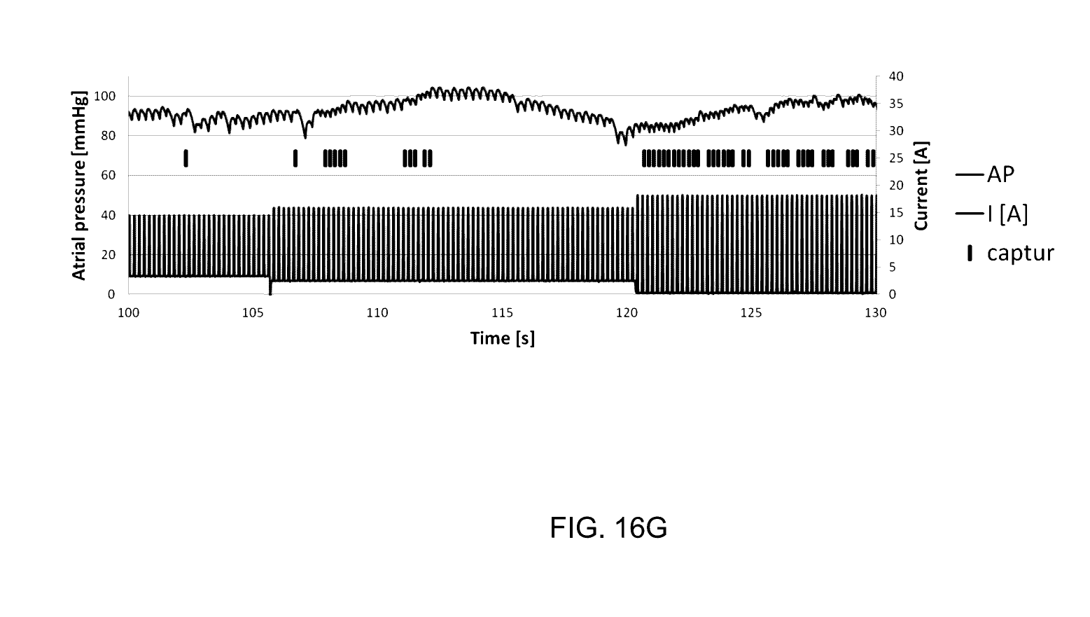

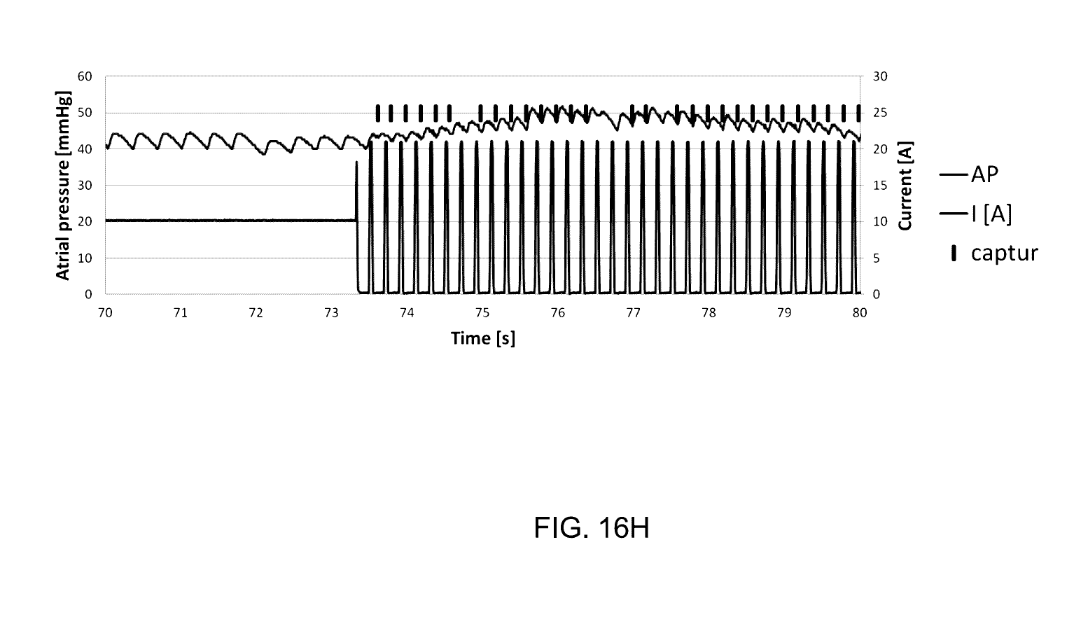

The effects of the pulses on the heart rhythm for Experiment Nos. 1-8 are demonstrated in FIGS. 16A-H, respectively. Shown are the current in the electromagnet coil (red line) and the AP (blue line), as a function of the time. The black marks at the top part of each of the graphs in FIGS. 16A-H designate heart beats that are synchronized with the pulses generated by the electromagnet.

### Magnetic Microparticles (MMP) Synthesis and Characterization

Several experiments were performed to increase the saturation magnetization of the synthesis of MMPs. Generally, magnetite nanoparticles are synthesized via alkaline precipitation of two iron salts—ferrous and ferric chloride (FeCl₂ and FeCl₃, respectively). However, the type of the alkaline solution (mostly sodium hydroxide or ammonium hydroxide) and its usage amount differ in different protocols, thus resulting in different magnetic properties of the magnetite.

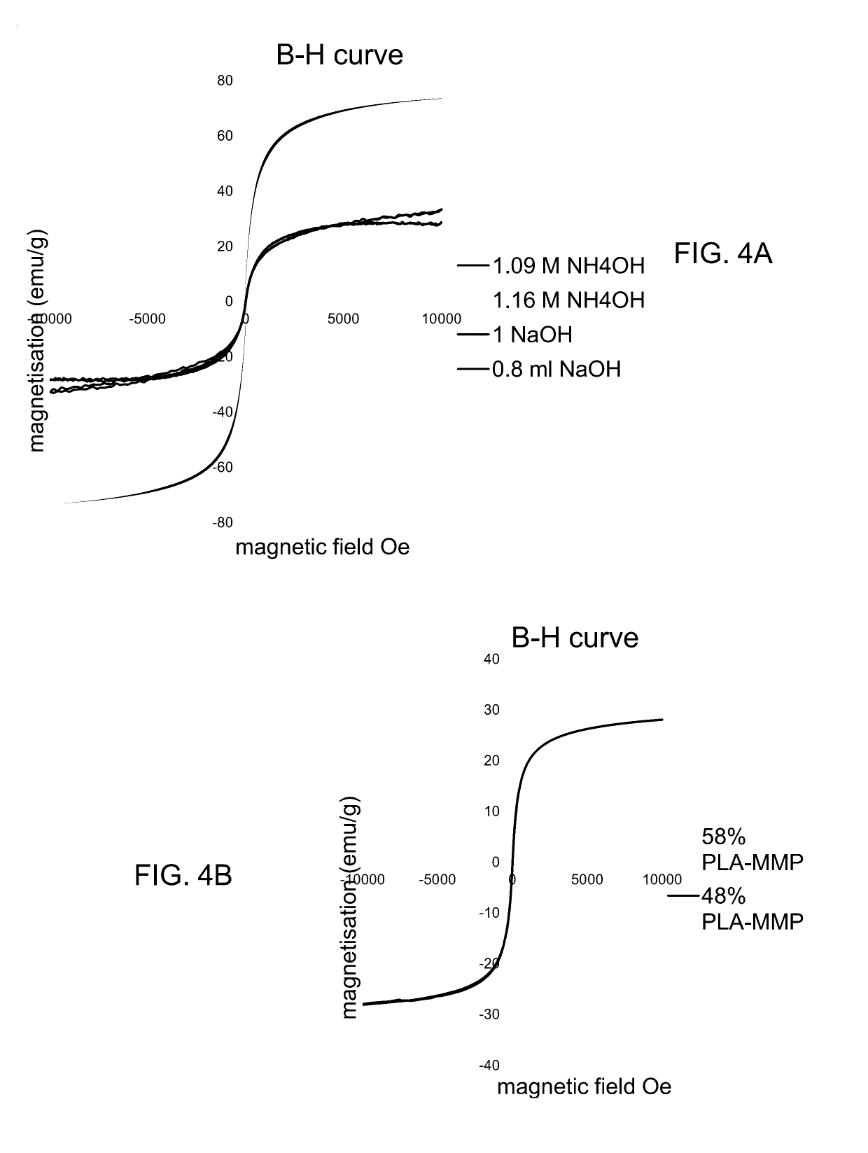

Iron salts were precipitated using different amount of sodium hydroxide or ammonium hydroxide and comparing the precipitated magnetite properties in a VSM device. FIGS. 3 and 4 show the different magnetic properties of magnetite obtained by different synthesis protocols, where FIG. 3 shows saturation magnetization values of magnetite synthesized in different amounts of ammonium hydroxide or sodium hydroxide, and MMPs containing 48% or 58% magnetite, and FIGS. 4A-B show hysteresis loop of (FIG. 4A) magnetite synthesized in different amounts of ammonium hydroxide or sodium hydroxide, and (FIG. 4B) PLA-coated MMPs with different magnetite percentage.

As shown, precipitation with ammonium hydroxide results in magnetite nanoparticles with the highest magnetization saturation values. This is probably due to optimal conditions that results in high yield of magnetite (which the iron oxide with the highest saturation magnetization reported) versus other iron oxides. The amount of ammonium hydroxide added did not affect dramatically the magnetic properties in the reported quantities. The addition of 0.66 ml or 2.5 ml NaOH, however, resulted in a brown suspension (rather than black magnetite) with almost no magnetic responsiveness, and the addition of 0.91 ml resulted in a black suspension with low magnetic responsiveness. Thus, in some embodiments of the present invention ammonium hydroxide is used as the precipitation solution using an amount of 20 ml ammonium hydroxide for 11.6 g ferric chloride which falls in the range of 0.76-0.81 ml.

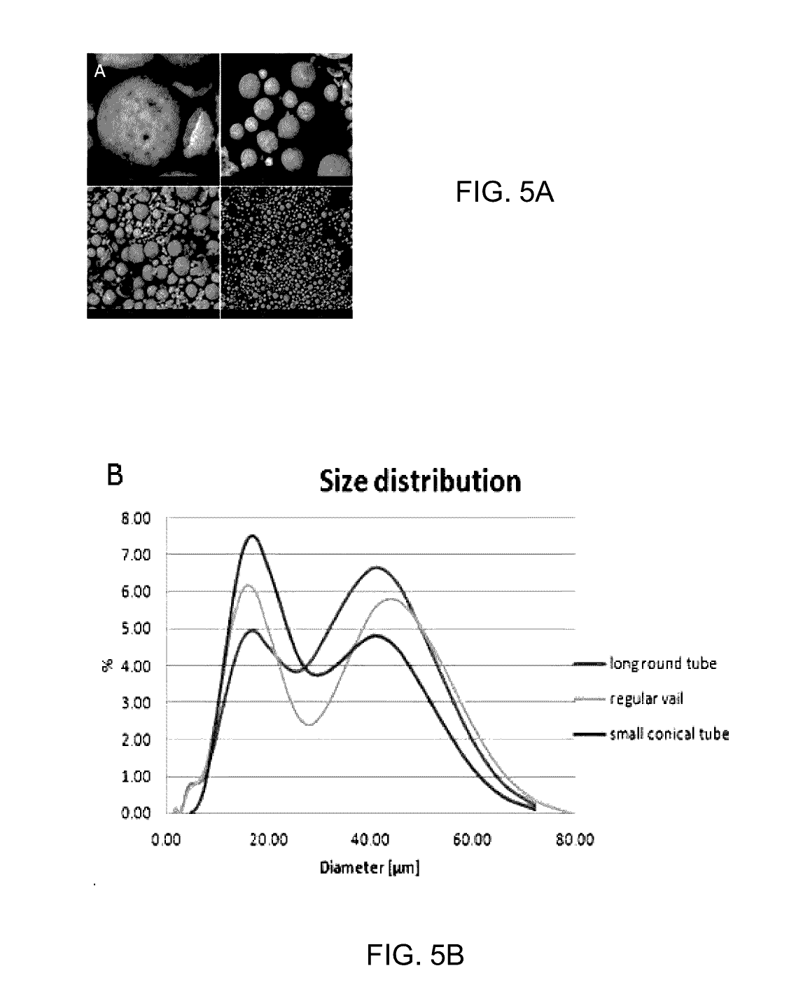

Using a double phase emulsion the lipophilic MNP was coated with PLA in order to obtain MMP in a size range that will allow their capture in the RV under blood flow. The emulsion was performed by vortexing the two phases in three different containers. The three different containers turned out to give approximately the same size distribution differing mainly in the height of the peaks and not in their location as illustrated in FIGS. 5A-B which show SEM images of the MMPs (FIG. 5A), and size distribution of the MMP as analyzed using SLS (FIG. 5B).

When comparing it to the SEM images, were no particle over 50 μm is found it seems that the large peak, at about 45 μm is due to particle aggregation and not a single particle.

### Theoretical Calculation of Particle Size

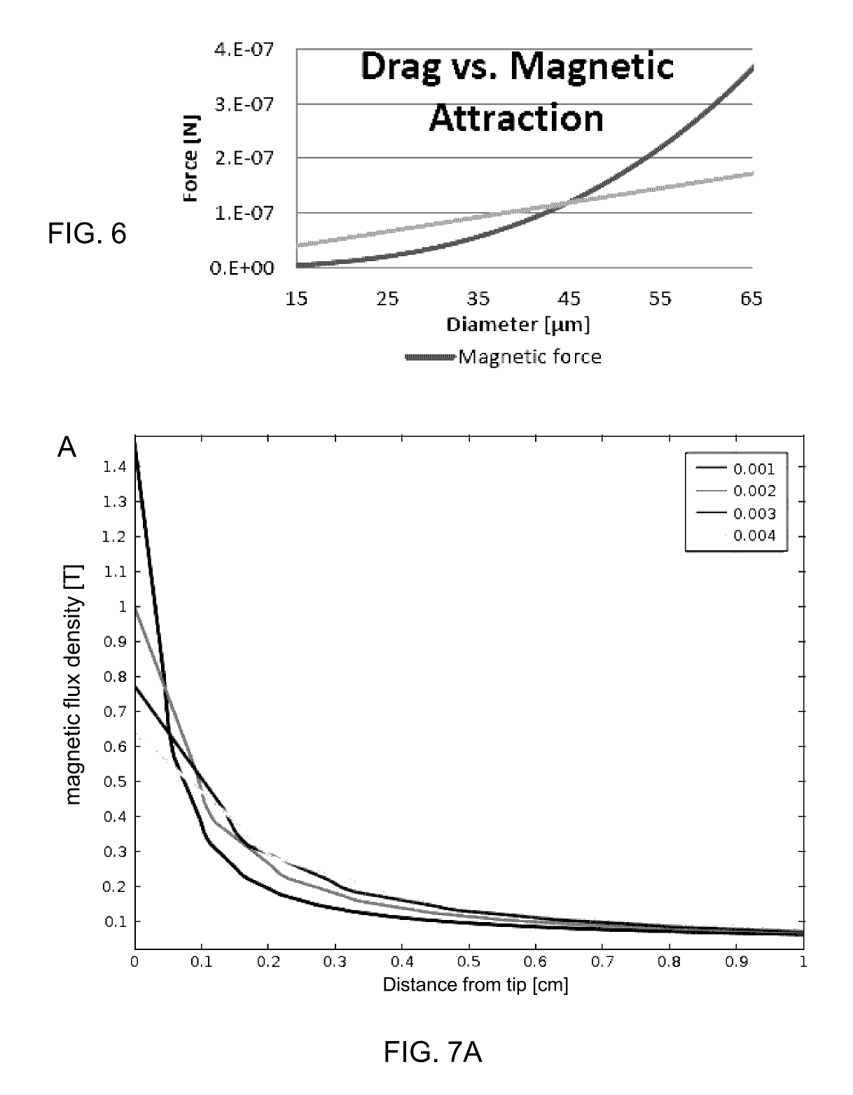

As explained above the drag force acting on a particle is proportional to the radius (EQ. 3) while the magnetic force is proportional to the third power of the radius (EQ. 2). FIG. 6 compares drag force to magnetic force as a function of particle size.

As shown, a particle size that exceeds 45 μm is subjected to magnetic force that is larger than the drag force applied by the blood flow. As for the blood velocity, 8 cm/s is typically the maximum speed in the RV. However, most of the volume of the RV which homogenously contains MMPs has smaller velocities. Additionally, the reported velocity is for diastole, which is the time of blood filling in the heart and therefore, the blood velocity is the highest during a full heartbeat. Furthermore, this theoretical assessment does not consider the interactions between particles. Under a magnetic field, each MMP becomes magnetized; therefore, the particles tend to aggregate when they are subjected to an external magnetic field. This aggregation results in larger particles so they are dominated by the magnetic force even if each particle alone is smaller than the theoretical limit of 45 μm. Thus, the theoretical assessment is not to be understood as an exact size limitation, but rather to provide a preferred order of magnitude of MMP size.

### Electromagnet Design

Generally, magnetic targeting studies are performed by using, in most cases, a permanent magnet as the magnetic gradient source. In some cases, however, electromagnet are used so that the field can be adjusted by current and different pole pieces to adapt the gradient to different sizes and shapes of target or they can be switched off for safety reasons.

The general concept of the designed electromagnet is that by inserting a steel core that narrows at its tip into the coil the magnetic field stream lines are concentrated and the magnetic flux density dramatically increases. A theoretical model of the electromagnet was constructed using COMSOL® Multiphysics software and different shapes of the tip.

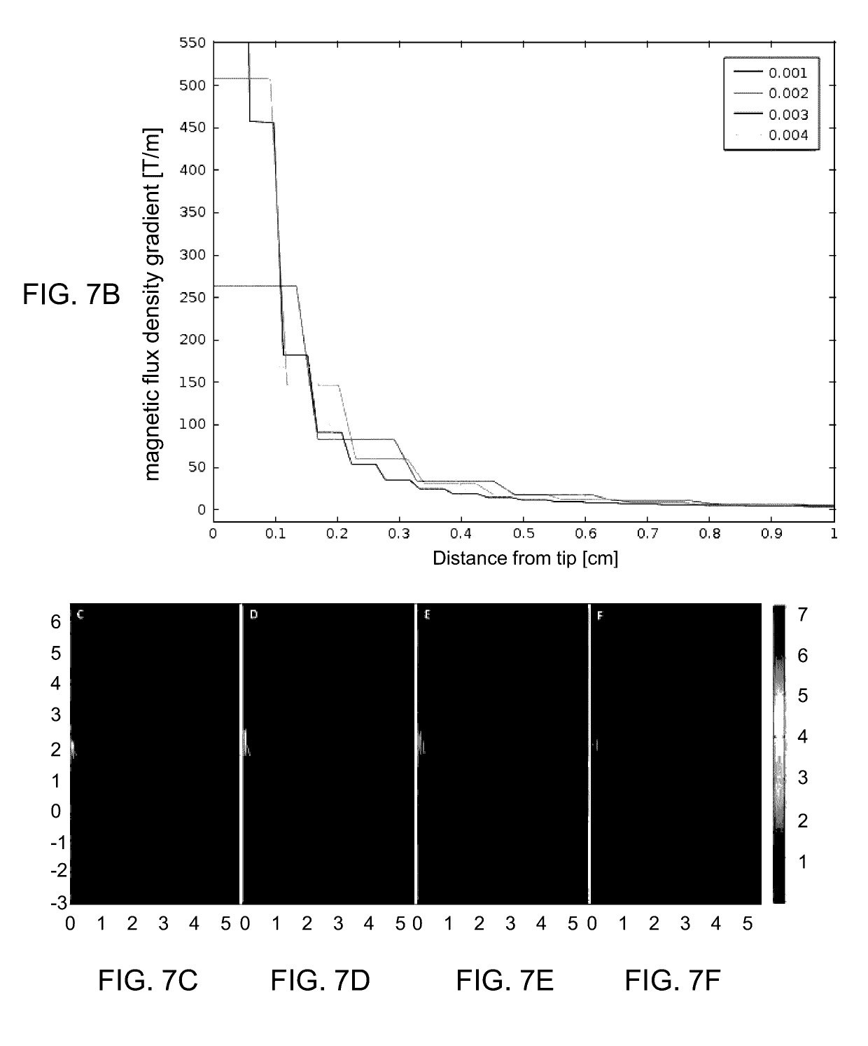

FIGS. 7A-F show the absolute magnetic flux density (FIG. 7A), the absolute magnetic flux density gradient versus distance from the coil tip (FIG. 7B), and the magnetic flux density intensities (color scale in T) and streamlines for tip diameter of 1, 2, 3 and 4 mm (FIGS. 7C, 7D, 7E and 7F, respectively), for the first electromagnet design.

The optimization criteria were high magnetic field (for high magnetization of the MMPs) and high magnetic field gradient. Another feature was the distance from the tip. While narrow tip size (about 1 mm) resulted in high values of both magnetic field and magnetic field gradient at the tip end they both rapidly decreased distant from the tip end (as shown by the streamlines that rapidly diverge in FIGS. 7C-F). Wide tip size (about 4 mm), however, resulted in lower levels of magnetic field and magnetic field gradient at the tip end, but they both decreased more moderately so that their values distant from the tip end were larger than for the small tip end (FIGS. 7A-B).

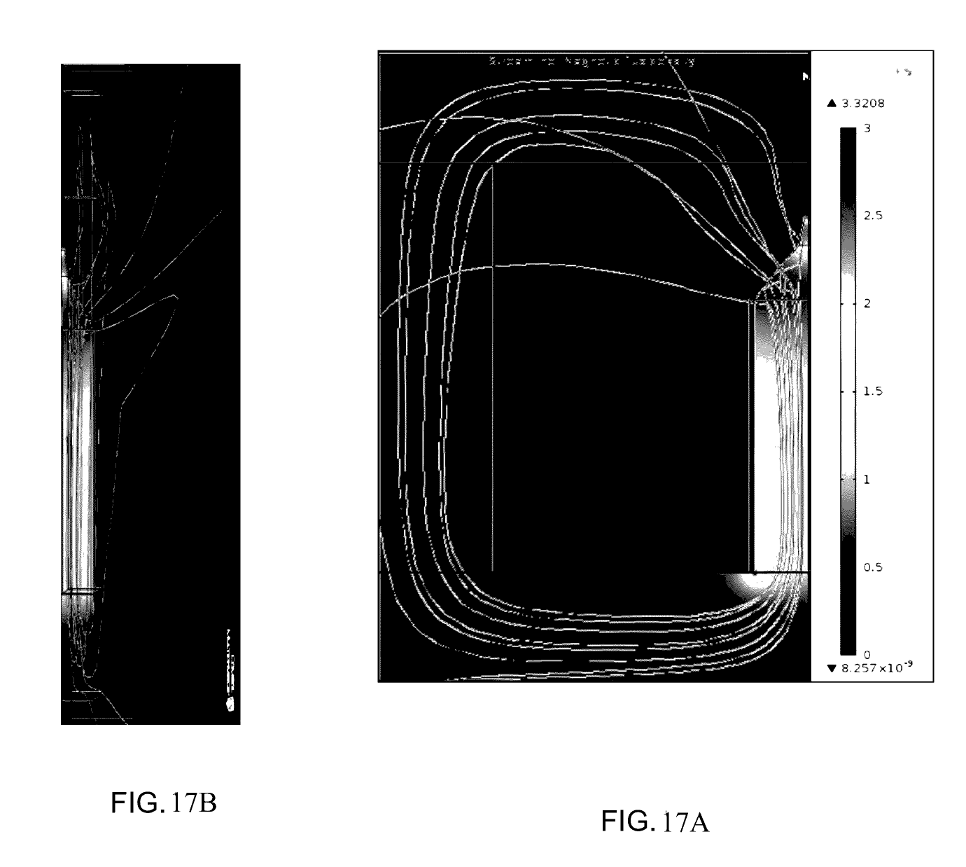

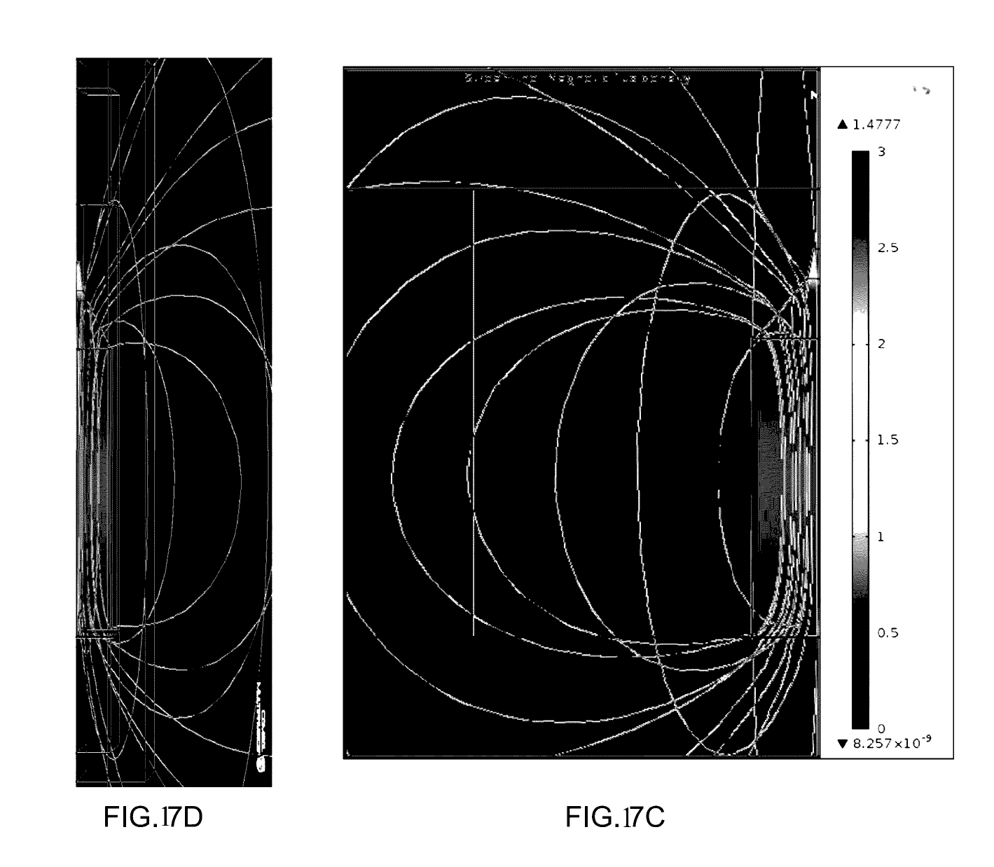

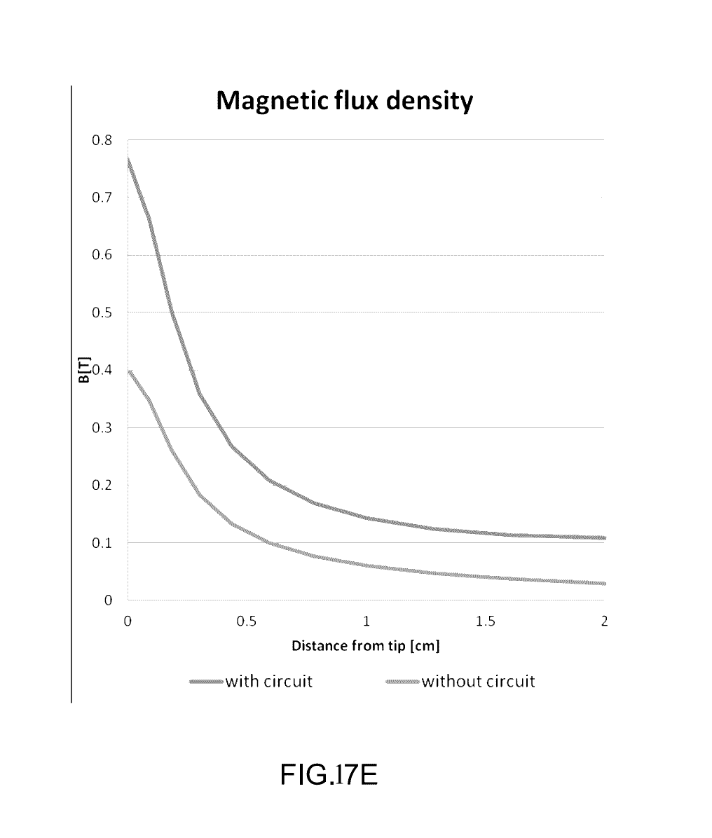

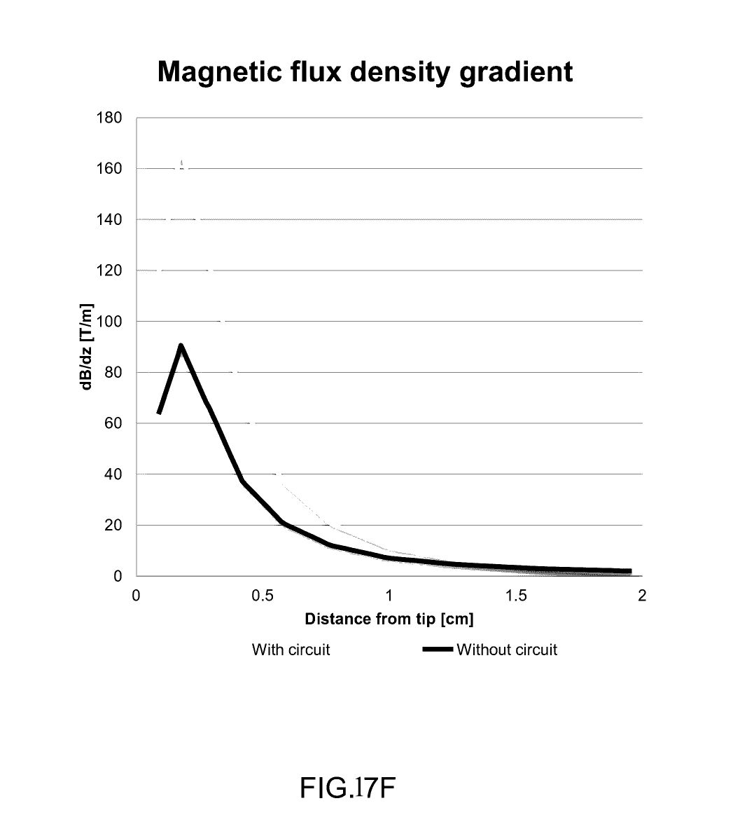

FIGS. 17A-F show the magnetic flux density intensities (color scale in T) and streamlines for the electromagnet with (FIGS. 17A and 17B) and without (FIGS. 17C and 17D) the metal loop forming a magnetic circuit, and the absolute magnetic flux density (FIG. 17E) and flux density gradient (FIG. 17F) as a function of the distance from the coil tip. FIGS. 17A and 17C show streamlines in a plane parallel to the metal loop, and FIGS. 17A and 17C show streamlines in a plane perpendicular to the metal loop.

### In Vitro RV Model

The in vitro RV model is constructed using several different angles (0°, 45° and) 90° and distances (1 and 5 mm). For each configuration different amounts of MMPs are injected. It is estimated that for each configuration there is a maximal amount of MMPs that may be captured in the RV model so that by plotting the weight of MMPs captured verses the amount injected a saturation curve is obtained.

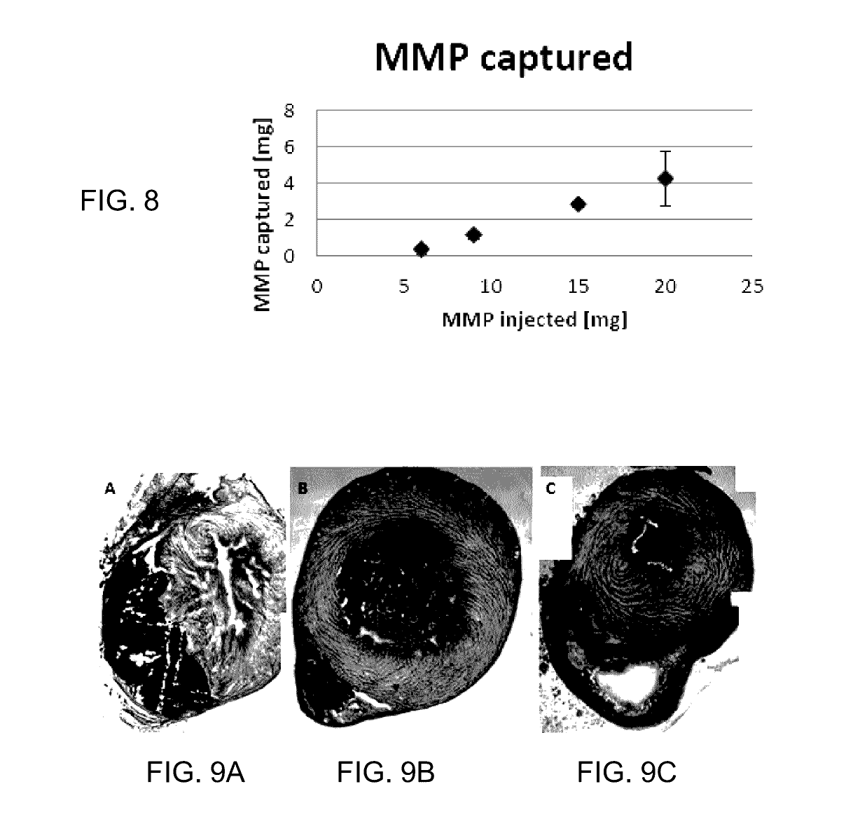

FIG. 8 shows data obtained from the in vitro RV model. As shown, the MMP pellet size saturation is not reached. For comparison, PLA encapsulated magnetite nano particles in the size range of 200-300 nm were also injected, and no MMPs were captured.

### MMPs Localization in a Rat RV

### Ultrasound Live Imaging of MMP Capture in the Heart

Ultrasound imaging was employed to view the magnetic particles passing the RV. After a clear image of the RV was obtained magnetic particles were injected into the rat tail vain. Immediately after injection the magnetic particles appeared in the RV as bright dots passing the image field. While smaller particles (200-300 nm) were not captured, magnetic microparticles remained in the RV showing as a bright interface on the interior wall of the ventricle. After approximately 30 seconds the magnet was removed from the chest and the ventricle image field again with bright dots. It is assumed that this is due to magnetic particle pellet that was held in place by the magnetic attraction released to the ventricle.

### Rat Model

Magnet was glued by tissue glue to the heart epicardium (in an open chest) or to the chest (in a closed chest) and 80 mg MMPs in 1 ml PBS were injected to the tail vain. After freezing and fixing the heart in OCT and cryo-sectioning the heart the section illustrated in FIG. 9 shows that MMPs were captured in the RV. FIG. 9A illustrates MMPs that are captured in the open chest rat after placing the magnet against the heart while (FIG. 9B) shows MMPs captured in a closed chest rat. It seems, that some of the MMPs escaped from the RV under blood flow and were recaptured in LV. FIG. 9C shows that when no magnet is positioned against the heart no significant amount of MMPs are localized in the RV.

### Use of Permanent Magnet

A small stainless steel bar was inserted into the RV of an anesthetized rat through the cardiac wall at the apical portion. The bar was remained attached to the wire in order to keep it in place avoiding it from being carried away by the blood flow. Then a neodymium magnet was manually positioned rapidly against the rat chest, inducing a pulse of magnetic attraction. Before this procedure the exposed heart was stimulated by gently touching the epicardium with a flexible stainless steel tip of an arterial line catheter guide wire.

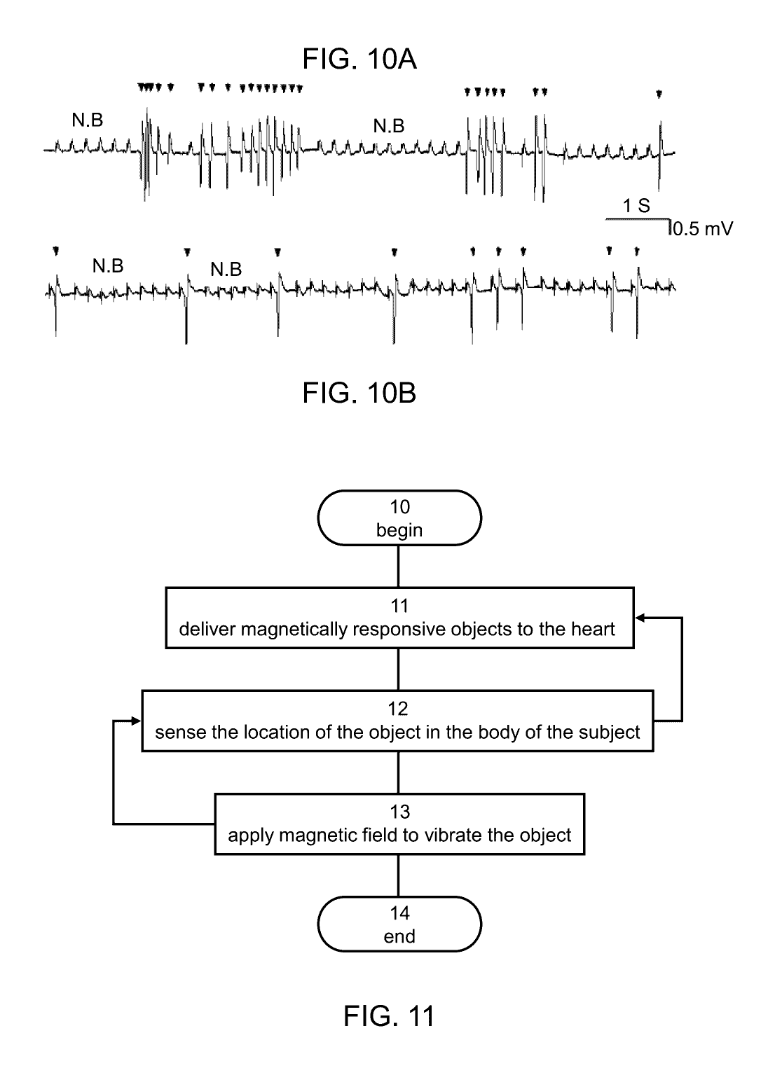

FIGS. 10A-B are ECG recordings showing mechanically induced pacing by gently touching the epicardium with a stainless steel tip (FIG. 10A), and by manually attracting the stainless steel bar on the endocardial surface of the rat heart. Arrow heads in FIGS. 10A-B indicate the beats generated by mechanical pacing (premature ventricular contractions). In FIGS. 10A-B the abbreviation N.B. means “normal beats”.

As shown, both stimulations provoked electrical pacing of the ventricles as indicated by the presence of premature ventricular contractions.

As used herein the term “about” refers to ±10%.

The word “exemplary” is used herein to mean “serving as an example, instance or illustration.” Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments.” Any particular embodiment of the invention may include a plurality of “optional” features unless such features conflict.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

[1] Dorwarth U, Frey B, Dugas M, Matis T, Fiek M, Schmoeckel M, Remp T, Durchlaub I, Gerth A, Steinbeck G, Hoffmann E. Transvenous defibrillation leads: High incidence of failure during long-term follow-up. Journal of cardiovascular electrophysiology. 2003; 14:38-43
[2] Cohen M I, Bush D M, Vetter V L, Tanel R E, Wieand T S, Gaynor J W, Rhodes L A. Permanent epicardial pacing in pediatric patients: Seventeen years of experience and 1200 outpatient visits. Circulation. 2001; 103:2585-2590
[3] Rossillo A, Verma A, Saad E B, Corrado A, Gasparini G, Marrouche N F, Golshayan A R, McCurdy R, Bhargava M, Khaykin Y, Burkhardt J D, Martin D O, Wilkoff B L, Saliba W I, Schweikert R A, Raviele A, Natale A. Impact of coronary sinus lead position on biventricular pacing: Mortality and echocardiographic evaluation during long-term follow-up. Journal of cardiovascular electrophysiology. 2004; 15:1120-1125
[4] Zoll P M, Zoll R H, Falk R H, Clinton J E, Eitel D R, Antman E M. External noninvasive temporary cardiac pacing: Clinical trials. Circulation. 1985; 71:937-944
[5] Corcoran S J, Davis L M. Cardiac implantable electronic device therapy for bradyarrhythmias and tachyarrhythmias. Heart, lung & circulation. 2012; 21:328-337
[6] Lee K L, Lau C P, Tse H F, Echt D S, Heaven D, Smith W, Hood M. First human demonstration of cardiac stimulation with transcutaneous ultrasound energy delivery: Implications for wireless pacing with implantable devices. Journal of the American College of Cardiology. 2007; 50:877-883
[7] Auricchio A, Delnoy P P, Regoli F, Seifert M, Markou T, Butter C. First-in-man implantation of leadless ultrasound-based cardiac stimulation pacing system: Novel endocardial left ventricular resynchronization therapy in heart failure patients. Europace: European pacing, arrhythmias, and cardiac electrophysiology: journal of the working groups on cardiac pacing, arrhythmias, and cardiac cellular electrophysiology of the European Society of Cardiology. 2013
[8] Kohl P, Ravens U. Cardiac mechano-electric feedback: Past, present, and prospect. Prog Biophys Mol Bio. 2003; 82:3-9
[9] Borneman. C, Scherf D. Paroxysmal ventricular tachycardia abolished by a blow to precordium. Dis Chest. 1969; 56:83-&
[10] Baderman H, Roberton N R. Thumping precordium. Lancet. 1965; 2:1293-&
[11] Barrett J S. Chest thumps and heart beat. New Engl J Med. 1971; 284:393-&
[12] Befeler B. Mechanical stimulation of heart—its therapeutic value in tachyarrhythmias. Chest. 1978; 73:832-838
[13] Lee H T, Cozine K. Incidental conversion to sinus rhythm from atrial fibrillation during external jugular venous catheterization. J Clin Anesth. 1997; 9:664-667
[14] Franz M R, Cima R, Wang D, Profitt D, Kurz R. Electrophysiological effects of myocardial stretch and mechanical determinants of stretch-activated arrhythmias. Circulation. 1992; 86:968-978
[15] Zoll P M, Belgard A H, Weintraub M J, Frank H A. External mechanical cardiac stimulation. The New England journal of medicine. 1976; 294:1274-1275
[16] Kohl P, Sachs F, Franz M R. Cardiac mechano-electric coupling and arrhythmias. Oxford; New York: Oxford University Press; 2011.
[17] Kohl P, Sachs F. Mechanoelectric feedback in cardiac cells. Philos T R Soc A. 2001; 359:1173-1185
[18] Kohl P, Sachs F, Franz M R. Cardiac mechano-electric feedback and arrhythmias: From pipette to patient. Philadelphia, Pa.: W.B. Saunders; 2005.
[19] Sensenig R, Sapir Y, MacDonald C, Cohen S, Polyak B. Magnetic nanoparticle-based approaches to locally target therapy and enhance tissue regeneration in vivo. Nanomedicine (Lond). 2012; 7:1425-1442
[20] Dames P, Gleich B, Flemmer A, Hajek K, Seidl N, Wiekhorst F, Eberbeck D, Bittmann I, Bergemann C, Weyh T, Trahms L, Rosenecker J, Rudolph C. Targeted delivery of magnetic aerosol droplets to the lung. Nature nanotechnology. 2007; 2:495-499
[21] Tseng P, Judy J W, Di Carlo D. Magnetic nanoparticle-mediated massively parallel mechanical modulation of single-cell behavior. Nature methods. 2012; 9:1113-1119
[22] Cooper P J, Epstein A, Macleod I A, Schaaf S T, Sheldon J, Boulin C, Kohl P. Soft tissue impact characterization kit (stick) for ex situ investigation of heart rhythm responses to acute mechanical stimulation. Progress in biophysics and molecular biology. 2006; 90:444-468
[23] Wen C, Li M, Whitworth J A. Validation of transonic small animal flowmeter for measurement of cardiac output and regional blood flow in the rat. Journal of cardiovascular pharmacology. 1996; 27:482-486

1. A method of pacing a heart of a subject, the heart having a magnetically responsive object therein, the method comprising non-invasively applying to the heart an alternating magnetic field selected to vibrate said object against a wall of the heart such as to effect mechanical stimulation of the heart, thereby pacing the heart.

2. The method of claim 1, further comprising delivering said magnetically responsive object to the heart.

3. The method of claim 1, wherein said delivering comprises injecting said object to the vasculature of the subject and noninvasively applying an external magnetic field to effect locomotion of said object within the vasculature and into the heart.

4. The method of claim 3, wherein said delivering comprises invasively implanting said object in the heart.

5. The method according to claim 1, wherein the object is in the cavity of a ventricle of the heart.

6. The method according to claim 5, wherein the object is in an apical portion of the cavity of a ventricle of the heart.

7. The method according to claim 5, wherein the object is in a right ventricle of the heart.

8. The method according to claim 5, wherein the object is in an apical portion of said right ventricle of the heart.

9. The method according to claim 1, wherein said magnetically responsive object comprises at least one of an injectable magnetic nanoparticle, an injectable magnetic microparticle, and a millimeter scale implantable device.

10. The method of claim 1, wherein said alternating magnetic field is alternating according to a waveform selected from the group consisting of sinusoidal waveform, square wave waveform, triangular waveform, ramp waveform, and any combination thereof.

11. The method of claim 1, further comprising sensing a location of the object in a body of the subject and/or the heart, wherein said application of said alternating magnetic field is responsive to said sensing.

12. The method of claim 11, wherein said sensing is by a pickup coil operative to generate voltage in response to a change in a magnetic flux.

13. The method according to claim 1, wherein said magnetic object comprises a magnetic material selected from the group consisting of a ferromagnetic material, a ferrimagnetic material and a superparamagnetic material.

14. The method according to claim 1, wherein said magnetic object comprises a superparamagnetic iron oxide.

15. The method according to claim 1, wherein said magnetic object comprises ferromagnetic iron microparticles.

16. The method according to claim 1, wherein said magnetic object comprises a magnetic particle coated by a lipophilic compound.

17. A system for pacing a heart of a subject, the heart having a magnetically responsive biocompatible object therein, the system comprises:
an electromagnet constituted to generate a magnetic field to which the biocompatible object is responsive, and
a controller configured to operate said electromagnet to generate an alternating magnetic field selected to vibrate said object once present in said heart against a wall of the heart such as to effect mechanical stimulation of the heart.

18. The system of claim 17, wherein said controller is configured to operate said electromagnet to generate said alternating magnetic field according to a waveform selected from the group consisting of sinusoidal waveform, square wave waveform, triangular waveform, ramp waveform, and any combination thereof.

19. The system according to claim 17, further comprising a pickup coil configured for sensing a location of the object in a body of the subject and/or the heart.

20. The system according to claim 17, further comprising a permanent magnet for applying a static magnetic field in addition to said alternating magnetic field.

21. A plurality of particles for use in a method for pacing a heart of a subject, the particles being biocompatible, introducible into the heart and responsive to a magnetic field, the method comprising non-invasively applying to the heart an alternating magnetic field selected to vibrate said particles once present in the heart against a wall of the heart such as to effect mechanical stimulation of the heart.

22. A plurality of particles for use in a method for pacing a heart of a subject, the particles being biocompatible, introducible into the heart and responsive to a magnetic field, the method being according to claim 1.
